(19) United States
(12) Patent Application Publication
(10) Pub. No.: US 2011/0052683 A1
(43) Pub. Date: Mar. 3, 2011

(54) PHARMACEUTICAL PREPARATION FOR TREATING CARDIOVASCULAR DISEASE

(75) Inventors: Sung Wuk Kim, Seongnam-si (KR); Sung Soo Jun, Seongnam-si (KR); Ja Seong Koo, Daejeon (KR); Young Joo Lee, Daejeon (KR); Seok Young Jang, Daejeon (KR); Young Gwan Jo, Daejeon (KR)

(73) Assignee: HANALL BIOPHARMA CO., LTD., Daejeon (KR)

(21) Appl. No.: 12/918,882

(22) PCT Filed: Feb. 21, 2009
(86) PCT No.: PCT/KR09/00803

§ 371 Date: Oct. 25, 2010

(30) Foreign Application Priority Data
Feb. 22, 2008 (KR) 10-2008-0016517

(51) Int. Cl. A61K 9/52; A61K 31/554; A61K 31/366; A61K 31/22; A61K 9/22; A61K 9/24; A61K 9/00; A61P 9/00; A61K 31/505; A61K 31/47; A61K 31/405

(52) U.S. Cl. 424/457; 514/211.09; 514/460; 514/510; 424/468; 424/472; 424/471; 424/400; 514/275; 514/311; 514/419

(57) ABSTRACT

The present invention provides a pharmaceutical preparation comprising a prior-release compartment containing a hydroxymethylglutaryl-CoA (HMG-CoA) reductase inhibitor as a pharmacologically active ingredient, and a delayed-release compartment containing a non-dihydropyridine calcium channel blocker as a pharmacologically active ingredient. The preparation of the present invention provides synergistic effects through combined administration of the non-dihydropyridine calcium channel blocker and the HMG-CoA reductase inhibitor, and induces the time-dependent absorption, metabolism and action mechanism of individual drugs through the controlled release thereof to avoid competitive antagonism between drugs, thus maximizing the effects of each pharmacologically active ingredient while minimizing side effects, for example, the risk of myopathy, and substantially increasing the compliance of patients by taking one tablet once a day.

### TECHNICAL FIELD

The present invention relates to a chronotherapeutic pharmaceutical preparation of a non-dihydropyridine calcium channel blocker and an HMG-CoA reductase inhibitor.

### BACKGROUND ART

Hypertension is a disease condition which is caused by a sustained high blood pressure outside a normal range, generally referring to a condition where a systolic blood pressure is 140 mmHg or higher or a diastolic blood pressure is 90 mmHg or higher. In South Korea, hypertension is a chronic circulatory disease with a high pathogenic incidence to an extent that one in five adults is suffering from such a condition. The incidence of hypertension is increasing all over the world. In addition, hypertension may cause fatal complications such as cerebral stroke, heart failure, and coronary artery diseases, even among minor or mild patients exhibiting no external symptoms. Therefore hypertension may require more active management and treatment for patients.

Meanwhile, hyperlipidemia is a condition where an excess of fat higher than a necessary level is present in blood and accumulates on blood vessel walls to cause inflammation, consequently resulting in cardiovascular diseases. Hyperlipidemia generally refers to the condition where a total cholesterol level is higher than 240 mg/dL or a triglyceride level is 200 mg/dL or higher. Hyperlipidemia may be a primary pathogenic cause for coronary artery diseases and may aggravate symptoms of hypertension.

Since arteriosclerosis and hypertension due to hyperlipidemia may aggravate symptoms of each other in a vicious circle as described above, there has been known that worsening of the disease in hypertensive patients can be prevented by simultaneous treatment of both atherosclerosis and hypertension [Hypertens Res 2001; 24: 3-11, and Hypertens Res 2003; 26: 1-36]. Thus, there has been a great deal of clinical results reporting synergistic effects of co-administration of a calcium channel blocker exhibiting anti-hypertensive effects and an HMG-CoA reductase inhibitor exhibiting cholesterol synthesis inhibitory effects.

Combined administration of these drugs is expected to provide synergistic effects, because the non-dihydropyridine calcium channel blocker serves as a therapeutic drug for hypertension as well as for arrhythmia and angina pectoris, and the HMG-CoA reductase inhibitor drug not only serves as a lipid-lowering agent, but also exhibits an anti-inflammatory activity on blood vessel walls or the like, thus also having anti-hypertensive effects [Cardiology 1992; 80 (Suppl 1): S31-S36, J Cardiovasc Pharmacol 1988; 12 (Suppl 7): S110-S113, Lancet 2000; 356: 359-365, Hypertens Res 2002; 25: 717-725, Hypertens Res 2002; 25: 329-333: Am J Manag Care. 2004 October; 10 (11 Suppl): S332-8: and Curr Control Trials Cardiovasc Med 2000, 1:161-165].

However, co-administration of these drugs may result in decreased drug efficiency of two drug groups and increased side effects thereof, since the non-dihydropyridine calcium channel blocker group inhibits the production of cytochrome P450 3A4 which is necessary for the activation of the HMG-CoA reductase inhibitor in the liver. In fact, according to Laerb Database, there has been a report showing muscle-related side effects by co-administration of an HMG-CoA reductase inhibitor drug and diltiazem or verapamil which is a non-dihydropyridine calcium channel blocker.

More particularly, diltiazem, a representative one of the non-dihydropyridine calcium channel blocker drugs, is metabolized via N-demethylation by cytochrome P450 (CYP450) in the liver and can potentially form N-desmethyl diltiazem. N-desmethyl diltiazem may form a metabolite intermediate complex with cytochrome P450 3A4 expressed by a cDNA in a microsome of the liver, to provide stronger inhibition of the production of cytochrome P450 3A4 than diltiazem, during diltiazem therapy is continued [Br. J. Clin. Pharmacol. 1997; 282: 294-300]; and J. Pharmacol. Exp. Ther. 1999, 290, 1116-1125].

For example, a co-administration of diltiazem and simvastatin exhibited a 3.5-fold increase in maximum blood concentration (Cₘₐₓ) of simvastatin and a 5-fold increase in area under the curve (AUC), as compared to single administration of simvastatin [Clinical Pharmacology & Therapeutics, 2000; 67: 267-274]. Further, there has been reported the occurrence of rhabdomyolysis and hepatitis when diltiazem is additionally administered during the medication of simvastatin [Tenn Med. 2001; 94:339-341].

Verapamil, another representative non-dihydropyridine calcium channel blocker drug, is also a cytochrome P450 3A4 inhibitor and has been found to be capable of changing the concentration of simvastatin through a cultivation of the liver microsome [Br. J. Clin. Pharmacol, 2001; 51: 461-470). In fact, a combined administration of verapamil and simvastatin exhibited a 2.6-fold increase in Cₘₐₓ of simvastatin, a 4.6-fold increase in AUC, a 3.4-fold increase in Cₘₐₓ of the simvastatin metabolite, and a 2.8-fold increase in AUC [Clin. Pharmacol. Ther. 1998, August 64(2), 177-182].

As discussed above, co-administration of a non-dihydropyridine drug (such as diltiazem or verapamil) and simvastatin results in an excessively increased blood concentration of simvastatin, which consequently deteriorates inhibitory effects of simvastatin on the cholesterol biosynthesis and causes serious side effects such as myolysis. These results may apply to lovastatin, atorvastatin, pravastatin and the like, which undergo a metabolic pathway similar to that of simvastatin.

For example, lovastatin is recommended to be administered at a dose of 40 mg/day or lower to avoid high daily dose because combined administration of lovastatin with verapamil may increase the risk of rhabdomyolysis and muscle-related side effects [MEVACOR Insert Manual], and it is known that care should be taken upon combined administration of lovastatin with diltiazem because combined administration of lovastatin with diltiazem leads to a sudden increase in Cₘₐₓ and AUC [Clin. Pharmacol. Ther. 1998; 64(4): 369-377]. Further, it is known that care should be taken upon combined administration of atorvastatin because combined administration of atorvastatin with diltiazem exhibited the occurrence of rhabdomyolysis and acute hepatitis [Ann Pharmacother. 2002; 36: 1546-1549]. Further, it is known that combined administration of pravastatin with verapamil exhibits 42% and 31% increases in Cₘₐₓ and AUC, respectively [Am. J. Cardiol. 2004; 94: 1140-1146].

To this end, the inventors of the present invention conducted extensive and intensive studies to develop an effective pharmaceutical preparation for treating cardiovascular diseases, which is capable of preventing mutual antagonism between drugs due to concurrent or combined administration of single drugs, and therefore completed the present invention.

### DISCLOSURE OF THE INVENTION

### Technical Problem

As a result of a variety of extensive and intensive studies and experiments in order to solve the problems as described above and to find a method which is capable of reducing side effects of drugs while enhancing clinical therapeutic effects upon combined administration thereof, the inventors of the present invention have developed a pharmaceutical preparation containing an HMG-CoA reductase inhibitor, which is represented by simvastatin or atorvastatin, and a non-dihydropyridine calcium channel blocker, which is represented by diltiazem or verapamil, based on the idea that when gastrointestinal absorption and dissolution of these drugs are controlled at a certain time interval, excessive blood concentration elevation and accumulation of the HMG-CoA reductase inhibitor can be inhibited to thereby prevent side effects of the drugs. The present invention has been completed based on these findings.

Therefore, the present invention provides a controlled-release pharmaceutical preparation of a controlled-release non-dihydropyridine calcium channel blocker and an HMG-CoA reductase inhibitor for functional chronotherapeutic administration.

Further, the present invention provides a controlled-release pharmaceutical preparation comprising diltiazem and atorvastatin, which is capable of reducing side effects of drugs while enhancing clinical therapeutic effects upon combined administration thereof.

### Technical Solution

The present invention provides a pharmaceutical preparation comprising a prior-release compartment containing a hydroxymethylglutaryl-CoA (HMG-CoA) reductase inhibitor as a pharmacologically active ingredient, and a delayed-release compartment containing a non-dihydropyridine calcium channel blocker as a pharmacologically active ingredient.

The preparation in accordance with the present invention provides more useful therapeutic effects by the provision of a physical compartment for controlling release properties between two active ingredients, thereby improving problems of conventional combined administration or concurrent administration of single drugs.

The present invention provides a pharmaceutical preparation wherein 85% or more of a total amount of the HMG-CoA reductase inhibitor in the preparation is released within one hour after initiation of the release thereof. In the preparation of the present invention, 80% or more of a total amount of the HMG-CoA reductase inhibitor in the preparation is preferably released within 30 minutes after initiation of the release thereof.

Further, the present invention provides a pharmaceutical preparation wherein the release of the non-dihydropyridine calcium channel blocker is initiated one hour after the release of the HMG-CoA reductase inhibitor is initiated, and may be finished within 24 hours. In the preparation of the present invention, more preferably, the release of the non-dihydropyridine calcium channel blocker may be initiated 2 hours after the release of the HMG-CoA reductase inhibitor is initiated, and is finished within 24 hours.

The present invention provides a pharmaceutical preparation wherein the non-dihydropyridine calcium channel blocker is released at a level of 40% or less of a total amount of a non-dihydropyridine calcium channel blocker in a unit preparation within 6 hours after the release of the HMG-CoA reductase inhibitor is initiated.

The present invention provides a pharmaceutical preparation wherein the release of drugs may be controlled such that the non-dihydropyridine calcium channel blocker may be absorbed by the liver 2 to 4 hours later than the HMG-CoA reductase inhibitor.

The HMG-CoA reductase inhibitor may include at least one selected from simvastatin, lovastatin, atorvastatin, pitavastatin, rosuvastatin, fluvastatin, pravastatin, pharmaceutically acceptable salts thereof and isomers thereof. The HMG-CoA reductase inhibitor may preferably include at least one selected from simvastatin, lovastatin, atorvastatin, pharmaceutically acceptable salts thereof and isomers thereof.

The non-dihydropyridine calcium channel blocker refers to a non-dihydropyridine calcium channel blocker which inhibits the production of a cytochrome P450 enzyme, and for example, may include at least one selected from diltiazem, verapamil, gallopamil, cinnarizine, flunarizine, isomers thereof and pharmaceutically acceptable salts thereof. The non-dihydropyridine calcium channel blocker may preferably include at least one selected from diltiazem, verapamil, isomers thereof and pharmaceutically acceptable salts thereof.

Further, the present invention may provide a pharmaceutical preparation including a prior-release compartment containing simvastatin, an isomer thereof or a pharmaceutically acceptable salt thereof as a pharmacologically active ingredient, and a delayed-release compartment containing diltiazem, an isomer thereof or a pharmaceutically acceptable salt thereof as a pharmacologically active ingredient.

Further, the present invention may provide a pharmaceutical preparation including a prior-release compartment containing lovastatin, an isomer thereof or a pharmaceutically acceptable salt thereof as a pharmacologically active ingredient, and a delayed-release compartment containing diltiazem, an isomer thereof or a pharmaceutically acceptable salt thereof as a pharmacologically active ingredient.

Further, the present invention may provide a pharmaceutical preparation including a prior-release compartment containing atorvastatin, an isomer thereof or a pharmaceutically acceptable salt thereof as a pharmacologically active ingredient, and a delayed-release compartment containing diltiazem, an isomer thereof or a pharmaceutically acceptable salt thereof as a pharmacologically active ingredient.

In the present invention, 85% or more of a total amount of atorvastatin in the preparation may be preferably released within one hour after release initiation thereof. Preferably, 85% or more of a total amount of atorvastatin in the preparation may be released within 30 minutes, and preferably 15 minutes after release initiation thereof.

Further, in the present invention, the release of diltiazem may be initiated one hour after release initiation of atorvastatin, preferably 2 hours after release initiation of atorvastatin, and may be finished within 24 hours.

The present invention may provide a pharmaceutical preparation wherein 20% or less, preferably 10% or less of a total amount of diltiazem in the unit preparation may be released within 5 hours after release initiation of atorvastatin. Further, in the present invention, 70% or more of diltiazem may be preferably released within 12 hours after release initiation of atorvastatin.

Further, the present invention may provide a pharmaceutical preparation including a prior-release compartment containing simvastatin, an isomer thereof or a pharmaceutically acceptable salt thereof as a pharmacologically active ingredient, and a delayed-release compartment containing verapamil, an isomer thereof or a pharmaceutically acceptable salt thereof as a pharmacologically active ingredient.

Further, the present invention may provide a pharmaceutical preparation including a prior-release compartment containing pravastatin, an isomer thereof or a pharmaceutically acceptable salt thereof as a pharmacologically active ingredient, and a delayed-release compartment containing verapamil, an isomer thereof or a pharmaceutically acceptable salt thereof as a pharmacologically active ingredient.

Hereinafter, individual ingredients of the pharmaceutical preparation in accordance with the present invention will be described in more detail.

1. Prior-Release Compartment

The prior-release compartment refers to a compartment which is released ahead of a delayed-release compartment in the pharmaceutical preparation of the present invention. The prior-release compartment contains (1) a pharmacologically active ingredient, and if necessary, (2) pharmaceutically acceptable additives.

(1) Pharmacologically Active Ingredients

As the pharmacologically active ingredient of the prior-release compartment, an HMG-CoA reductase inhibitor may include at least one selected from simvastatin, lovastatin, atorvastatin, pitavastatin, rosuvastatin, fluvastatin, pravastatin, pharmaceutically acceptable salts thereof and isomers thereof. The HMG-CoA reductase inhibitor may preferably include simvastatin, lovastatin, atorvastatin, or pravastatin.

In accordance with the preparation of the present invention, a dose of the HMG-CoA reductase inhibitor, which is a pharmacologically active ingredient in the prior-release compartment, may be in a range of 0.1 to 160 mg/day, preferably 1 to 80 mg/day, based on the preparation (a total of 200 to 1200 mg), for an adult (adult male weighing 65 to 75 kg).

(2) Pharmaceutically Acceptable Additives

The preparation of the present invention may further contain commonly used additives such as pharmaceutically acceptable diluent, binder, disintegrant, lubricant, pH-adjusting agent, stabilizer, and solubilizer, within a range where effects of the present invention are not impaired and the release of the pharmacologically active ingredients is not impaired.

Examples of the diluent may include starch, microcrystalline cellulose, lactose, glucose, mannitol, alginate, an alkaline earth metal salt, clay, polyethylene glycol, dicalcium phosphate, and a mixture thereof.

In the prior-release compartment of the present invention, a content of the additive may be in a range of 0.1 to 300 parts by weight, relative to 1 part by weight of the HMG-CoA reductase inhibitor.

Examples of the diluent that may be used in the prior-release compartment of the present invention may include starch, microcrystalline cellulose, lactose, glucose, mannitol, alginate, an alkaline earth metal salt, clay, polyethylene glycol, dicalcium phosphate, and a mixture thereof.

Examples of the binder that may be used in the prior-release compartment of the present invention may include starch, microcrystalline cellulose, highly dispersive silica, mannitol, sucrose, lactose, polyethylene glycol, polyvinylpyrrolidone, hydroxypropylmethylcellulose, hydroxypropylcellulose, natural gum, synthetic gum, copovidone, povidone, gelatin, or a mixture thereof.

Examples of the disintegrant that may be used in the prior-release compartment of the present invention may include starches or modified starches such as sodium starch glycolate, corn starch, potato starch, and pregelatinized starch, clays such as bentonite, montmorillonite, and veegum, celluloses such as microcrystalline cellulose, hydroxypropylcellulose, and carboxymethylcellulose, algins such as sodium alginate, and alginic acid, crosslinked celluloses such as croscarmellose sodium, gums such as guar gum, and xanthan gum, crosslinked polymers such as crosslinked polyvinylpyrrolidone (crospovidone), effervescent formulations such as sodium bicarbonate and citric acid, or mixtures thereof.

Examples of the lubricant that may be used in the prior-release compartment of the present invention may include talc, stearic acid, magnesium stearate, calcium stearate, sodium lauryl sulfate, hydrogenated vegetable oil, sodium benzoate, sodium stearyl fumarate, glyceryl behenate, glyceryl monooleate glyceryl monostearate, glyceryl palmitostearate or polyethylene glycol.

Examples of the pH-adjusting agent that may be used in the prior-release compartment of the present invention may include acidulants such as acetic acid, adipic acid, ascorbic acid, malic acid, succinic acid, tartaric acid, fumaric acid, and citric acid, alkalizing agents such as precipitated calcium carbonate, aqueous ammonia, and meglumine or mixture thereof.

Examples of the stabilizer that may be used in the prior-release compartment of the present invention may include alkalizers such as alkali metal salts, alkaline earth metal salts, or mixtures thereof. Examples of the alkali metal salts and alkaline earth metal salts may include calcium carbonate, sodium carbonate, sodium hydrogen carbonate, magnesium oxide, magnesium carbonate, sodium citrate. etc.

Examples of the solubilizer that may be used in the prior-release compartment of the present invention may include sodium lauryl sulfate, polyoxyethylene sorbitan fatty acid ester (such as polysorbate), docusate sodium, etc.

In addition, the preparation of the present invention may optionally contain pharmaceutically acceptable additives such as various additives selected from a colorant and a fragrance. The range of the additive that may be used in the present invention may not be limited to the above-mentioned additives, and the additive may be used in a conventionally used dose.

2. Delayed-Release Compartment

In the present invention, the delayed-release compartment refers to a compartment whose active ingredient is released at a certain time interval after the release of the active ingredient of the prior-release compartment. The delayed-release compartment contains (1) a non-dihydropyridine calcium channel blocker which is a pharmacologically active ingredient and (2) a release-controlling material or (3) an osmo-regulator and a semi-permeable membrane coating base, and if necessary, (4), pharmaceutically acceptable additives.

(1) Pharmacologically Active Ingredients

The pharmacologically active ingredient of the delayed-release compartment refers to a non-dihydropyridine calcium channel blocker which inhibits the production of a cytochrome P450 enzyme, and examples thereof include, but are not limited to, diltiazem, verapamil, gallopamil, cinnarizine, flunarizine, isomers thereof and pharmaceutically acceptable salts thereof.

Preferred examples of the non-dihydropyridine calcium channel blocker may include diltiazem, verapamil, isomers thereof or pharmaceutically acceptable salts.

In the present invention, a dose of the non-dihydropyridine calcium channel blocker may be in a range of 10 to 500 mg/day, preferably 20 to 420 mg/day based on the preparation (a total of 200 to 1300 mg), for an adult (adult male weighing 65 to 75 kg).

(2) Release-Controlling Materials

The release-controlling material in the pharmaceutical preparation of the present invention may include at least one release-controlling material selected from an enteric polymer, a water-insoluble polymer, a hydrophobic compound, a hydrophilic polymer and a mixture thereof. The release-controlling material in the pharmaceutical preparation may preferably include at least one selected from the enteric polymer and the water-insoluble polymer.

In the delayed-release compartment of the present invention, a content of the release-controlling material may be in a range of 0.01 to 100 parts by weight relative to 1 part by weight of the non-dihydropyridine calcium channel blocker. When a content of the release-controlling material is lower than 0.01 parts by weight, it may be difficult to achieve a sufficient time-lag. On the other hand, when a content of the release-controlling material is higher than 100 parts by weight, there is a problem associated with no release of the drug or excessively delayed release having a time-lag of 9 hours or more.

In the delayed-release compartment of the present invention, the enteric polymer refers to a polymer which is insoluble or stable under acidic conditions having an acidity less than pH 5, and is dissolved or degraded under predetermined pH conditions of pH 5 or higher.

In the present invention, the enteric polymer may include one selected from the group consisting of an enteric cellulose derivative, an enteric acrylic acid copolymer, an enteric maleic acid copolymer, an enteric polyvinyl derivative, and a mixture thereof.

The enteric cellulose derivative may include at least one selected from hydroxypropylmethylcellulose acetate succinate, hydroxypropylmethylcellulose phthalate, hydroxymethylethylcellulose phthalate, cellulose acetate phthalate, cellulose acetate succinate, cellulose acetate maleate, cellulose benzoate phthalate, cellulose propionate phthalate, methylcellulose phthalate, carboxymethylethylcellulose, ethylhydroxyethylcellulose phthalate, methylhydroxyethylcellulose and a mixture thereof; the enteric acrylic acid copolymer may include at least one selected from a styrene/acrylic acid copolymer, a methyl acrylate/acrylic acid copolymer, a methyl acrylate/methacrylic acid copolymer, a butyl acrylate/styrene/acrylic acid copolymer, a methacrylic acid/methyl methacrylate copolymer (e.g., Eudragit L 100 or Eudragit S, Degussa, Germany), a methacrylic acid/ethyl acrylate copolymer (e.g., Eudragit L 100-55, Degussa, Germany), a methyl acrylate/methacrylic acid/octyl acrylate copolymer and a mixture thereof; the enteric maleic acid copolymer may include at least one selected from a vinyl acetate/maleic anhydride copolymer, a styrene/maleic anhydride copolymer, a styrene/maleic monoester copolymer, a vinyl methyl ether/maleic anhydride copolymer, an ethylene/maleic anhydride copolymer, a vinyl butyl ether/maleic anhydride copolymer, an acrylonitrile/methyl crylate/maleic anhydride copolymer, a butyl acrylate/styrene/maleic anhydride copolymer and a mixture thereof; and the enteric polyvinyl derivative may preferably include at least one selected from polyvinylalcohol phthalate, polyvinylacetal phthalate, polyvinylbutyrate phthalate, polyvinylacetacetal phthalate and a mixture thereof.

A content of the enteric polymer may be in a range of 0.01 parts by weight to 10 parts by weight, preferably 0.08 parts by weight to 0.4 parts by weight relative to 1 part by weight of the non-dihydropyridine calcium channel blocker. When a content of the enteric polymer is lower than 0.01 parts by weight, it is difficult to achieve a sufficient time-lag. On the other hand, when a content of the enteric polymer is higher than 10 parts by weight, it is difficult to achieve significant effects as a time-lag is longer than desired.

In the delayed-release compartment of the present invention, the water-insoluble polymer refers to a pharmaceutically acceptable water-insoluble polymer which controls the release of a drug. The water-insoluble polymer that may be used in the present invention may preferably include at least one selected from the group consisting of polyvinyl acetate, a polymethacrylate copolymer, a poly(ethyl acrylate, methyl methacrylate) copolymer, a poly(ethyl acrylate, methyl methacrylate, trimethylaminoethyl methacrylate) copolymer, ethylcellulose, cellulose ester, cellulose ether, cellulose acylate, cellulose diacylate, cellulose triacylate, cellulose acetate, cellulose diacetate, cellulose triacetate and a mixture thereof. The water-insoluble polymer may more preferably include a poly(ethyl acrylate, methyl methacrylate, trimethylaminoethyl methacrylate) copolymer.

A content of the water-insoluble polymer may be in a range of 0.01 parts by weight to 10 parts by weight, preferably 0.05 parts by weight to 1.25 parts by weight relative to 1 part by weight of the non-dihydropyridine calcium channel blocker. When a content of the water-insoluble polymer is lower than 0.01 parts by weight, it is difficult to achieve a sufficient time-lag. On the other hand, when a content of the water-insoluble polymer is higher than 10 parts by weight, there is a problem associated with no release of the drug or excessively delayed release having a time-lag of 9 hours or more.

In the delayed-release compartment of the present invention, the hydrophobic compound refers to a pharmaceutically acceptable water-insoluble material which controls the release of a drug. The hydrophobic compound that may be used in the present invention may include at least one selected from the group consisting of fatty acid and fatty acid ester, fatty acid alcohol, wax, an inorganic material, and a mixture thereof.

The fatty acid or fatty acid ester may preferably include at least one selected from glyceryl palmitostearate, glyceryl stearate, glyceryl behenate, cetyl palmitate, glyceryl monooleate, stearic acid and a mixture thereof the fatty acid alcohol may include at least one selected from cetostearyl alcohol, cetyl alcohol, stearyl alcohol and a mixture thereof the wax may include at least one selected from carnauba wax, beeswax, microcrystalline wax and a mixture thereof and the inorganic material may include at least one selected from talc, precipitated calcium carbonate, calcium hydrogen phosphate, zinc oxide, titanium oxide, kaolin, bentonite, montmorillonite, veegum and a mixture thereof.

A content of the hydrophobic compound may be in a range of 0.01 parts by weight to 10 parts by weight, preferably 0.05 parts by weight to 0.5 parts by weight relative to 1 part by weight of the non-dihydropyridine calcium channel blocker. When a content of the hydrophobic compound is lower than 0.01 parts by weight, it is difficult to achieve a sufficient time-lag release. On the other hand, when a content of the hydrophobic compound is higher than 10 parts by weight, it is impossible to achieve significant clinical effects due to delayed release of the drug.

In the delayed-release compartment of the present invention, the hydrophilic polymer refers to a pharmaceutically acceptable water-soluble polymer which controls the release of a drug. The hydrophilic polymer that may be used in the present invention may include at least one selected from the group consisting of saccharide, a cellulose derivative, gum, a protein, a polyvinyl derivative, a polymethacrylate copolymer, a polyethylene derivative, a carboxyvinyl copolymer and a mixture thereof. Preferably, the saccharide may include at least one selected from dextrin, polydextrin, dextran, pectin and a pectin derivative, alginate, polygalacturonic acid, xylan, arabinoxylan, arabinogalactan, starch, hydroxypropyl starch, amylose, amylopectin and a mixture thereof; the cellulose derivative may include at least one selected from hydroxypropylmethylcellulose, hydroxypropylcellulose, hydroxymethylcellulose, hydroxyethylcellulose, methylcellulose, sodium carboxymethylcellulose, hydroxypropylmethylcellulose acetate succinate, hydroxyethylmethylcellulose and a mixture thereof; the gum may include at least one selected from guar gum, locust bean gum, tragacanth, carrageenan, gum acacia, gum arabic, gellan gum, xanthan gum and a mixture thereof; the protein may include at least one selected from gelatin, casein, zein and a mixture thereof; the polyvinyl derivative may include at least one selected from polyvinyl alcohol, polyvinyl pyrrolidone, polyvinylacetal diethylaminoacetate and a mixture thereof; the polymethacrylate copolymer may include at least one selected from a poly(butyl methacrylate, (2-dimethylaminoethyl)methacrylate, methyl methacrylate) copolymer, a poly(methacrylic acid, methyl methacrylate) copolymer, a poly(methacrylic acid, ethyl acrylate) copolymer and a mixture thereof; the polyethylene derivative may include at least one selected from polyethylene glycol, polyethylene oxide and a mixture thereof; and the carboxyvinyl polymer may include carbomer.

A content of the hydrophilic polymer may be in a range of 0.01 parts by weight to 10 parts by weight, preferably 0.14 to 0.7 parts by weight relative to 1 part by weight of the non-dihydropyridine calcium channel blocker. When a content of the hydrophilic polymer is lower than 0.01 parts by weight, it may be difficult to achieve a sufficient time-lag. On the other hand, when a content of the hydrophilic polymer is higher than 10 parts by weight, it may be difficult to achieve significant effects due to a time-lag of 9 hours or more.

In the preparation of the present invention, the delayed-release material may preferably include at least one selected from polyethylene oxide, polyvinyl acetate, a poly(ethyl acrylate, methyl methacrylate, trimethylaminoethyl methacrylate) copolymer, a polymethacrylate copolymer, a polymethyl methacrylate ethyl acrylate copolymer, a carboxyvinyl polymer, hydroxypropylmethylcellulose phthalate, titanium oxide and a mixture thereof.

(3) Osmo-Regulator and Semi-Permeable Membrane Coating Base

The delayed-release compartment of the present invention may be a compartment which contains an osmo-regulator and is coated by a semi-permeable membrane coating base.

In the delayed-release compartment of the present invention, the osmo-regulator may preferably include at least one selected from the group consisting of magnesium sulfate, magnesium chloride, sodium chloride, lithium chloride, potassium sulfate, sodium sulfate, lithium sulfate and a mixture thereof.

A content of the osmo-regulator may be in a range of 0.01 parts by weight to 10 parts by weight, preferably 0.05 parts by weight to 0.5 parts by weight relative to 1 part by weight of the non-dihydropyridine calcium channel blocker. When a content of the osmo-regulator is lower than 0.01 parts by weight, it may be difficult to achieve a sufficient chronotherapeutic release. On the other hand, when a content of the osmo-regulator is higher than 10 parts by weight, it is impossible to achieve significant clinical effects due to delayed release of a drug.

In the present invention, the semi-permeable membrane coating base may be a material which included in a coating layer of the pharmaceutical preparation. The semi-permeable membrane coating base refers to a material used to form a membrane through which some ingredients may pass but other ingredients may not pass. The semi-permeable coating base in the present invention may include the above-mentioned water-insoluble polymers.

A content of the semi-permeable membrane coating base may be in a range of 0.01 parts by weight to 10 parts by weight, preferably 0.05 parts by weight to 1.25 parts by weight relative to 1 part by weight of the non-dihydropyridine calcium channel blocker. When a content of the semi-permeable membrane coating base is lower than 0.01 parts by weight, it may be difficult to achieve a sufficient time-lag. On the other hand, when a content of the semi-permeable membrane coating base is higher than 10 parts by weight, there is a problem associated with no release of the drug or an excessively long time-lag of 9 hours or more.

(4) Pharmaceutically Acceptable Additives

In addition to (2) the release-controlling material and (3) the osmo-regulator and semi-permeable membrane coating base, the preparation of the present invention may further contain commonly used additives such as pharmaceutically acceptable diluent, binder, disintegrant, lubricant, pH-adjusting agent, anti-foaming agent, and solubilizer, within the range where the effects of the present invention are not impaired and within the range where delayed-release properties are not compromised.

Examples of the diluent that may be used in the present invention may include starch, microcrystalline cellulose, lactose, glucose, mannitol, alginate, an alkaline earth metal salt, clay, polyethylene glycol, dicalcium phosphate, and a mixture thereof. Examples of the binder that may be used in the present invention may include starch, microcrystalline cellulose, highly dispersive silica, mannitol, sucrose, lactose, polyethylene glycol, polyvinylpyrrolidone, hydroxypropylmethylcellulose, hydroxypropylcellulose, natural gum, synthetic gum, copovidone, povidone, gelatin, and a mixture thereof.

Examples of the disintegrant that may be used in the present invention may include starches or modified starches such as sodium starch glycolate, corn starch, potato starch, and pregelatinized starch, clays such as bentonite, montmorillonite, and veegum, celluloses such as microcrystalline cellulose, hydroxypropylcellulose, and carboxymethylcellulose, algins such as sodium alginate, and alginic acid, crosslinked, celluloses such as croscarmellose sodium, gums such as guar gum, and xanthan gum, crosslinked polymers such as crosslinked polyvinylpyrrolidone (crospovidone), effervescent formulations such as sodium bicarbonate and citric acid, and mixtures thereof.

Examples of the lubricant that may be used in the present invention may include talc, stearic acid, magnesium stearate, calcium stearate, sodium lauryl sulfate, hydrogenated vegetable oil, sodium benzoate, colloidal silicon dioxide, sodium stearyl fumarate, glyceryl behenate, glyceryl monooleate, glyceryl monostearate, glyceryl palmitostearate and polyethylene glycol.

As the pharmaceutically acceptable additive, examples of the pH-adjusting agent that may be used in the present invention may include acidulants such as acetic acid, adipic acid, ascorbic acid, malic acid, succinic acid, tartaric acid, fumaric acid, and citric acid, and alkalizing agents such as precipitated calcium carbonate, aqueous ammonia, and meglumine.

As the pharmaceutically acceptable additive of the present invention, examples of the anti-foaming agent may include dimethicone, oleyl alcohol, propylene glycol alginate, and simethicone (such as simethicone emulsion).

As the pharmaceutically acceptable additive of the present invention, examples of the solubilizer may include sodium lauryl sulfate, polyoxyethylene sorbitan fatty acid ester such as polysorbate, and docusate sodium.

In addition, the preparation of the present invention may include pharmaceutically acceptable additives such as various additives selected from a colorant and a fragrance. A range of the additives that may be used in the present invention may not be limited to the above-mentioned additives, and the additive may be used in a conventionally used dose.

Further, in the delayed-release preparation of the present invention, as a binding solvent and a solvent for the delayed-release additive, there may be employed purified water, ethanol, methylene chloride, and the like. More preferred may be purified water, and ethanol.

In the pharmaceutically acceptable additive of the present invention, a range of the usable additive may not be limited to the above-mentioned additives, and the additive may be used in a conventionally used dose.

The pharmaceutical preparation of the present invention may be prepared into various formulations, for example, tablets (such as uncoated tablets, coated tablets, multi-layered tablets, or press coated tablets), powders, granules, or capsules.

The pharmaceutical preparation of the present invention may be in the form of a two-phase matrix tablet including a delayed-release compartment and a prior-release compartment surrounding the delayed-release compartment.

Further, the pharmaceutical preparation of the present invention may be in the form of a film-coated tablet including a tablet containing a delayed-release compartment and a film-coating layer containing a prior-release compartment enclosing the exterior of the tablet, whereby atorvastatin of the film-coating layer may be first released as the film-coating layer is dissolved.

Further, the pharmaceutical preparation of the present invention may be in the form of a multi-layered tablet having a multi-layered structure of a delayed-release compartment and a prior-release compartment. The multi-layered tablet may be formed by mixing a granule constituting the delayed-release compartment with its own pharmaceutical additives and a granule constituting the prior-release compartment with its own pharmaceutical additives as well and then compressing the granules into a double-layered or triple-layered tablet using a multiple tablet press. The resulting preparation may be a tablet for an oral administration which is formulated to achieve the prior-release and delayed-release of drugs according to individual layers.

Further, the pharmaceutical preparation of the present invention may be in the form of a press coated tablet including an inner core containing a delayed-release compartment and an outer layer containing a prior-release compartment enclosing the outer surface of the inner core. The press coated tablet may be an osmotic press coated tablet. The osmotic press coated tablet may be a formulation wherein the tablet mix is compressed into a tablet in a manner that an osmo-regulator is incorporated into a inner compartment of the tablet for the delayed-release of a drug, the tablet surface is coated with a semi-permeable membrane coating base to prepare an inner core, and a granule constituting the prior-release compartment is mixed with a pharmaceutical additive to prepare an outer layer, followed by compression to form a formulation having a delayed-release inner core and a prior-release layer enclosing the surface of the inner core.

The pharmaceutical preparation of the present invention may be in the form of a capsule containing a particle, granule, pellet, or tablet containing a delayed-release compartment and a particle, granule, pellet, or tablet formed of a prior-release compartment.

The preparation of the present invention may further include a coating layer on the outside of the delayed-release compartment and/or the prior-release compartment. That is, the surface of particles, granules, pellets, or tablets containing the delayed-release compartment and/or the prior-release compartment may be coated for the purpose of controlled release of drugs or stability of the preparation.

Further, the pharmaceutical preparation of the present invention may be in the form of a kit including a delayed-release compartment and a prior-release compartment. Particularly, the present invention may be in the form of a kit wherein a particle, granule, pellet, or tablet constituting the prior-release compartment is prepared, a granule, pellet, or tablet constituting the delayed-release compartment is additionally prepared, and the thus prepared two release compartments are filled in a foil, blister, or bottle to prepare a dosage form for concurrent administration of different drugs.

The preparation of the present invention may be a preparation in the form of an uncoated tablet without further coating, or otherwise, if necessary, may be provided in the form of a coated tablet further including a coating layer formed on the outside of the preparation. The formation of a coating layer may provide a preparation which is capable of further securing stability of active ingredients.

A method for forming the coating layer may be suitably selected by a skilled person in the art, from among methods capable of forming a film-like coating layer on the surface of the tablet layer, such as a fluidized-bed coating method and a pan coating method. Preferably, a pan coating method may be used.

The coating layer may be prepared by using a film-forming agent, a film-forming aid or a mixture thereof. Particularly, the film-forming agent may include cellulose derivatives such as hydroxypropylmethylcellulose and hydroxypropylcellulose, saccharide derivatives, polyvinyl derivatives, waxes, fats, gelatin and mixtures thereof, and the film-forming aid may include polyethylene glycol, ethylcellulose, glyceride, titanium oxide, talc, diethyl phthalate and mixtures thereof.

A content of the coating layer may be in a range of 0.5 to 15% by weight (% w/w) based on the total weight of the tablet.

Further, the present invention provides a pharmaceutical preparation for evening administration in accordance with the present invention.

The preparation of the present invention may be administered once a day, particularly in the evening time (17 to 22-23 o'clock), thus providing maximized effects of individual active ingredients and minimized side effects. Generally, a biorhythm may be an important factor that is taken into consideration upon the treatment of patients suffering from hypertension, or hyperlipidemia. For example, the synthesis of lipids in the liver may be active after prior evening meals, and the blood pressure of normal persons including hypertensive patients may decline between night and dawn and begins to rise in the morning after awakening and peaks during the daytime (during activity). Upon considering these facts, when the preparation of the present invention is administered in the evening, an HMG-CoA reductase inhibitor, which is a prior-releasing active ingredient, may exhibit greater lipid-lowering effects since the HMG-CoA reductase inhibitor is administered at a time point when hepatic enzymes are activated, and the blood pressure after dawn may be effectively lowered by a non-dihydropyridine calcium channel blocker which is delayed-released, whereby the blood pressure may be uniformly maintained from dawn to morning. As a consequence, it is possible to avoid competitive antagonism between drugs and maximize effects of individual active ingredients.

The present invention provides a method for treating a cardiovascular disease, comprising administering the pharmaceutical preparation of the present invention to a mammal.

The cardiovascular disease may include hypertension and complications thereof of people suffering from a metabolic syndrome with combined manifestation of hypertension, or diabetes, obesity, hyperlipidemia, and coronary artery diseases.

The pharmaceutical preparation of the present invention may be preferably formulated into a desired dosage form depending on individual diseases or ingredients, by an appropriate method known in the art, for example, using the principle of the chronotherapy as disclosed in Chrontherapeutics (2003, Peter Redfern, PhP), particularly by a method including the following steps.

Step 1 may be a step of obtaining a delayed-release granule or tablet by subjecting a non-dihydropyridine calcium channel blocker and one or two release-controlling materials selected from the group consisting of an enteric polymer, a water-insoluble polymer, a hydrophobic compound, and a hydrophilic polymer together with a pharmaceutically acceptable conventional additive to mixing, kneading, drying, granulation or coating, and compression, or of obtaining a delayed-release granule or tablet by subjecting a non-dihydropyridine calcium channel blocker and an osmo-regulator together with a conventional pharmaceutically acceptable additive to mixing, kneading, drying, granulation or compression, followed by coating with a semi-permeable membrane coating base.

Step 2 may be a step of obtaining a prior-release granule or tablet by subjecting an HMG-CoA reductase inhibitor together with a conventional additive to conventional processes for producing oral solid preparations, for example, mixing, kneading, drying, granulation or coating, and compression.

Step 3 may be a step of obtaining a preparation for oral administration by mixing the granule or tablet obtained in each of Steps 1 and 2 with a pharmaceutically acceptable excipient and either compressing the mixture into a tablet or filling the mixture in a capsule for oral administration.

Step 1 may be carried out after Step 2, or Step 1 may be carried out simultaneously with Step 2.

The pharmaceutical preparation of the present invention may be prepared according to the above procedure, and a formulation method of Step 3 will be described in more detail hereinafter, but the present invention is not limited thereto.

1. Preparation of Two-Phase Matrix Tablets

The particles or granules prepared in Step 1 may be optionally coated with a release-controlling material and then mixed with the granules prepared in Step 2, followed by compression into a uniform weight, thereby preparing tablets. The resulting tablets may be film-coated for the purpose of improving a stability or shape, if necessary.

2. Preparation of Film-Coated Tablets containing Active Ingredients

The coated tablets or granules prepared in Step 1 may be optionally coated with a release-controlling material and dried, followed by compression into a uniform weight and optionally further coating to prepare tablets. In addition, an HMG-CoA reductase inhibitor may be dissolved and dispersed in a water-soluble film coating solution and may be coated on the outer layer of the tablets prepared in Step 1 to thereby prepare oral film-coated tablets containing active ingredients in the film coating.

3. Preparation of Multi-Layered Tablets

The granules prepared in Step 1 may be optionally coated with a release-controlling material, and dried. The dried granules may be compressed with the granules prepared in Step 2 by using a multi-layered tablet press, thereby obtaining a double-layered tablet. According to the formulation design or if necessary, a triple or more multi-layered tablet may also be prepared by further adding a release adjuvant layer on the double-layered tablet. A coated multi-layered tablet may be prepared by coating the multi-layered tablet.

4. Preparation of Press Coated Tablets

The coated tablets or granules prepared in Step 1 may be optionally coated with a release-controlling material and dried, followed by compression into a uniform weight. The resulting tablet may be used as an inner core optionally after performing further coating, and compressed with the granules prepared in Step 2 by using a press coated tableting press, thereby providing press coated tablets in where the surface of the tablet of Step 1 may be enclosed by the prior-release layer. Coated press coated tablets may be prepared by coating the press coated tablets.

5. Preparation of Capsules (Containing Granules or Tablets)

The granules prepared in Step 1 may be optionally coated with a release-controlling material, and dried. The dried granules together with the granules prepared in Step 2 may be placed in a capsule filling machine, and filled in capsules having a given size at an effective amount of each main ingredient, thereby preparing capsules.

6. Preparation of Capsules (Pellets)

(1) A non-dihydropyridine calcium channel blocker, a release-controlling material, and if necessary, pharmaceutically acceptable additives may be dissolved or suspended in water, an organic solvent, or a mixed solvent. This solution or suspension may be coated on sugar spheres and dried, and if necessary, coated with one or more release-controlling materials dissolved in water, an organic solvent, or a mixed solvent, followed by drying. The mixture may be mixed with the granules prepared in Step 2 or the tablets obtained in Step 3 and then filled in capsules using a capsule filling machine, thereby preparing capsules.

(2) An HMG-CoA reductase inhibitor and pharmaceutically acceptable additives may be dissolved or suspended in water, an organic solvent or a mixed solvent, coated on sugar spheres, followed by drying, and mixed with the controlled-release pellets of Section (1) containing a non-dihydropyridine calcium channel blocker and filled in capsules using a capsule filling machine to prepare capsules.

7. Preparation of Kit

The non-dihydropyridine calcium channel blocker-containing preparation obtained in Step 1 and the HMG-CoA reductase inhibitor-containing preparation obtained in Step 2 may be filled in a foil, blister, or bottle to prepare a kit for concurrent administration of different drugs.

### Advantageous Effects

As described above, the present invention provides a pharmaceutical preparation which is designed based on chronotherapeutics and xenobiotics for maximizing therapeutic effects and for pharmacodynamically improving side effects that may occur upon combination use of different drugs. The combination product of the present invention comprises, as active ingredients, a non-dihydropyridine calcium channel blocker and a statin-based lipid-reducing agent, which are affected by the same cytochrome P450 enzyme or inhibit enzyme activity. At the same time, the combination comprises a release-controlling material, which may control the time when the active ingredients are released in a body, such that the pharmacologically active ingredients may be released at different times and different release rates in the body.

Therefore, the preparation of the present invention may provide synergistic effects through combined administration of a non-dihydropyridine calcium channel blocker and an HMG-CoA reductase inhibitor, and induce the time-dependent absorption, metabolism and action mechanism of individual drugs through the controlled release thereof to avoid competitive antagonism between drugs, thus maximizing the effects of each pharmacologically active ingredient while minimizing side effects, for example, the risk of myopathy, and substantially increasing the compliance of patients by taking one tablet once a day.

### DESCRIPTION OF THE DRAWINGS

FIG. 1 is a graph illustrating the comparative dissolution profiles of a pharmaceutical preparation of diltiazem/simvastatin prepared in Example 1, and control drugs (Zocor: simvastatin single drug, Cardizem LA: diltiazem single drug).

FIG. 2 is a graph illustrating the comparative dissolution profiles of pharmaceutical preparations of diltiazem/simvastatin prepared in Examples 7 and 10, and control drugs (Zocor: simvastatin single drug, Cardizem CD: diltiazem single drug).

FIG. 3 is a graph illustrating the comparative dissolution profiles of a pharmaceutical preparation of diltiazem/lovastatin prepared in Example 11, and control drugs (Mevacor: lovastatin single drug, Cardizem LA: diltiazem single drug).

FIG. 4 is a graph illustrating the comparative dissolution profiles of a pharmaceutical preparation of verapamil/simvastatin prepared in Example 23, and control drugs (Zocor: simvastatin single drug, Isoptin SR: verapamil single drug).

FIG. 5 is a graph illustrating the comparative dissolution profiles of a pharmaceutical preparation of verapamil/pravastatin prepared in Example 28, and control drugs (Zocor: simvastatin single drug, Pravachol: pravastatin single drug).

FIG. 6 is a graph illustrating the comparative dissolution profiles of a pharmaceutical preparation of diltiazem/atorvastatin prepared in Example 30, and control drugs (Lipitor: atorvastatin single drug, Cardizem LA: diltiazem single drug).

FIG. 7 is a graph illustrating the comparative dissolution profiles of pharmaceutical preparations of diltiazem/atorvastatin prepared in Examples 37 and 45, and control drugs (Lipitor: atorvastatin single drug, Cardizem CD: diltiazem single drug).

### MODE FOR INVENTION

Advantages and features of the present invention and methods of achieving the same will become apparent from the detailed embodiments given below. This invention may, however, be embodied in different forms and should not be construed as being limited to the embodiments set forth herein. Rather, these embodiments are provided so that this disclosure will be thorough and complete, and will fully convey the scope of the invention to those skilled in the art. Therefore, the present invention should be defined by attached claims only.

### Example 1

### Preparation of Diltiazem-Simvastatin Two-Phase Matrix Tablets

(1) Preparation of Diltiazem Delayed-Release Granules

According to the ingredients and contents illustrated in Table 1 below, diltiazem hydrochloride, fumaric acid and hydroxypropylmethylcellulose were sieved through a No. 35 sieve, and mixed in a double cone mixer (Dasan Pharmatech, South Korea). The mixture was placed in a high-speed mixer (Lab. Pharma Mixer P, Diosna, Germany) and Kollicoat SR30D was added thereto, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve to prepare the title delayed-release granules.

(2) Preparation of Simvastatin Prior-Release Granules

According to the ingredients and contents illustrated in Table 1 below, simvastatin, microcrystalline cellulose, and D-mannitol were sieved through a No. 35 sieve and mixed in a high-speed mixer to prepare a mixture of main ingredients. Meanwhile, hydroxypropylcellulose and citric acid were dissolved in purified water to prepare a binding solution. The binding solution and the mixture of main ingredients were placed in a high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve. Butylated hydroxyanisole was mixed with the sieved material to prepare the title prior-release granules.

(3) Post-Mixing, Compression and Coating

The final compositions prepared in Processes (1) and (2) were mixed in a double cone mixer. The mixture was mixed with sodium starch glycolate and colloidal silicon dioxide given in a prior-release compartment of Table 1 and finally mixed with magnesium stearate in a double cone mixer.

The final mixture was compressed into tablets using a rotary tablet press (MRC-30: Sejong, South Korea). Using a coating solution prepared by mixing coating layer materials given in Table 1, a film-coating layer was formed on the tablets by a Hi-coater, thereby preparing the title two-phase matrix tablets.

### Example 2

### Preparation of Diltiazem-Simvastatin Two-Phase Matrix Tablets

(1) Preparation of Diltiazem Delayed-Release Granules

According to the ingredients and contents illustrated in Table 1 below, diltiazem hydrochloride, fumaric acid and hydroxypropylmethylcellulose were sieved through a No. 35 sieve and mixed in a double cone mixer. The mixture was placed in a fluidized bed granulator (GPCG 1: Glatt). Meanwhile, ethylcellulose was dissolved in 200 mg of ethanol to prepare a binding solution which was then sprayed thereon to form granules, followed by drying. A solution of Eudragit RS PO in a 1:1 (450 mg:450 mg) mixture of ethanol and methylene chloride was sprayed and coated on the granules to prepare the title granules.

(2) Preparation of Simvastatin Prior-Release Granules

According to the ingredients and contents illustrated in Table 1 below, simvastatin, microcrystalline cellulose and D-mannitol were sieved through a No. 35 sieve and mixed in a high-speed mixer. Meanwhile, hydroxypropylcellulose and citric acid were dissolved in purified water to prepare a binding solution which was then kneaded with the mixture of main ingredients. After completion of the kneading process, the kneaded material was granulated using an oscillator with a No. 20 sieve, and the granules were dried in a hot-water dryer at 60° C. After completion of the drying process, the granules were sieved again through a No. 20 sieve. Butylated hydroxyanisole was mixed with the sieved material to prepare the title granules.

(3) Post-Mixing, Compression and Coating

Using the final products of processes (1) and (2), the title in the form of two-phase matrix tablets were prepared in the same procedure as in process (3) of example 1.

### Example 3

### Preparation of Diltiazem-Simvastatin Multi-Layered Tablets

(1) Preparation of Diltiazem Delayed-Release Layer

According to the ingredients and contents illustrated in Table 1 below, diltiazem hydrochloride, fumaric acid and hydroxypropylmethylcellulose were sieved through a No. 35 sieve and mixed in a double cone mixer. Meanwhile, ethylcellulose was dissolved in 200 mg of ethanol to prepare a binding solution which was then sprayed thereon to form granules, followed by drying. A solution of hydroxypropylmethylcellulose phthalate in a 1:1 (450 mg:450 mg) mixture of ethanol and methylene chloride was sprayed and coated on the resulting granules. Magnesium stearate was added thereto, followed by final mixing in a double cone mixer to prepare the title delayed-release layer.

(2) Preparation of Simvastatin Prior-Release Layer

According to the ingredients and contents illustrated in Table 1 below, simvastatin, microcrystalline cellulose, and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were placed in a high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve. Butylated hydroxyanisole, sodium starch glycolate, and colloidal silicon dioxide were mixed with the sieved material and magnesium stearate was added thereto, followed by final mixing in a double cone mixer to prepare the title prior-release layer.

(3) Compression and Coating

In a multilayer tablet press (MRC-37T, Sejong, South Korea), the final simvastatin-containing composition of Process (2) was placed in a first powder feeder, and the final diltiazem-containing composition of Process (1) was placed in a second powder feeder. The compositions in the feeders were compressed into tablets under such conditions that the interlayer incorporation may be minimized Using a coating solution prepared by mixing coating layer ingredients given in Table 1, a film-coating layer was formed on the compressed tablets by a Hi-coater, thus preparing the title multi-layered tablets.

### Example 4

### Preparation of Diltiazem-Simvastatin Multi-Layered Tablets

(1) Preparation of Diltiazem Delayed-Release Layer

According to the ingredients and contents illustrated in Table 1 below, diltiazem hydrochloride, fumaric acid and hydroxypropylmethylcellulose were sieved through a No. 35 sieve and mixed in a double cone mixer. The mixture was placed in a fluidized bed granulator. Meanwhile, ethylcellulose was dissolved in ethanol to prepare a binding solution which was then sprayed thereon to form granules, followed by drying. A solution of hydroxypropylmethylcellulose phthalate in a 1:1 (450 mg:450 mg) mixture of ethanol and methylene chloride was sprayed and coated on the resulting granules. Magnesium stearate was added thereto, followed by final mixing in a double cone mixer to prepare the title delayed-release layer.

(2) Preparation of Simvastatin Prior-Release Layer

According to the ingredients and contents illustrated in Table 1 below, the title prior-release layer was prepared in the same procedure as in Process (2) of Example 3.

(3) Compression and Coating

Using the final compositions of Processes (1) and (2), the title sustained-release tablets in the form of a multi-layered tablet were prepared in the same procedure as in Process (3) of Example 3.

### Example 5

### Two-phase preparation of diltiazem-simvastatin capsules

(1) Preparation of Diltiazem Delayed-Release Pellets

According to the ingredients and contents illustrated in Table 1 below, diltiazem hydrochloride, fumaric acid, and hydroxypropylmethylcellulose were sieved through a No. 35 sieve and were placed together with sugar seeds (sugar spheres) in a fluidized bed granulator, followed by mixing. Meanwhile, hydroxypropylmethylcellulose was dissolved in purified water to prepare a binding solution. The binding solution was sprayed thereon to form diltiazem-containing pellets, followed by drying. A solution of hydroxypropylmethylcellulose phthalate in a 1:1 (450 mg:450 mg) mixture of ethanol and methylene chloride was sprayed on the granules to coat the pellets.

(2) Preparation of Simvastatin Prior-Release Granules

According to the ingredients and contents illustrated in Table 1 below, simvastatin, microcrystalline cellulose, and D-mannitol were sieved through a No. 35 sieve and mixed in a high-speed mixer. Meanwhile, hydroxypropylcellulose and citric acid were dissolved in purified water to prepare a binding solution which was then kneaded together with the mixture of main ingredients. After completion of the kneading process, the kneaded material was granulated using an oscillator with a No. 20 sieve, and the granules were dried in a hot-water dryer at 60° C. After completion of the drying process, the granules were sieved again through a No. 20 sieve, and butylated hydroxyanisole was added thereto, followed by mixing to prepare the title granules.

(3) Mixing and Capsule Filling

The final compositions of Processes (1) and (2) were mixed in a double cone mixer. The mixture was mixed with sodium starch glycolate given in a prior-release compartment of Table 1, using a double cone mixer, followed by mixing with colloidal silicon dioxide, and final mixing with magnesium stearate. The final mixture was placed in a powder feeder, and filled in two No. 1 gelatin hard capsules using a capsule filling machine to prepare two capsules each containing both 180 mg of diltiazem and 10 mg of simvastatin.

### Example 6

### Two-phase preparation of diltiazem-simvastatin capsules

(1) Preparation of Diltiazem Delayed-Release Pellets

According to the ingredients and contents illustrated in Table 1 below, diltiazem hydrochloride, fumaric acid, and hydroxypropylmethylcellulose were sieved through a No. 35 sieve and were placed together with sugar seeds (sugar spheres) in a fluidized bed granulator. Meanwhile, hydroxypropylmethylcellulose was dissolved in purified water to prepare a binding solution. The binding solution was sprayed thereon to form diltiazem-containing pellets, followed by drying. Kollicoat SR30D was sprayed and coated on the diltiazem-containing pellets to prepare pellets.

(2) Preparation of Simvastatin Prior-Release Tablets

According to the ingredients and contents illustrated in Table 1 below, simvastatin, microcrystalline cellulose, and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were placed in a high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve, and butylated hydroxyanisole was added thereto, followed by mixing. Sodium starch glycolate was added to the composition, followed by mixing in a double cone mixer, further mixing with colloidal silicon dioxide, and final mixing with magnesium stearate.

The final mixture was compressed to prepare the title prior-release tablets containing 10 mg of simvastatin per tablet, using a rotary tablet press (MRC-30: Sejong, South Korea).

(3) Capsule Filling

The final compositions of Processes (1) and (2) were filled in two No. 1 hydroxypropylmethylcellulose hard capsules using a capsule filling machine to prepare the title capsules each containing both 180 mg of diltiazem and 10 mg of simvastatin.

### Example 7

### Two-phase preparation of diltiazem-simvastatin capsules

(1) Preparation of Diltiazem Delayed-Release Granules

According to the ingredients and contents illustrated in Table 1 below, diltiazem hydrochloride, fumaric acid and hydroxypropylmethylcellulose were sieved through a No. 35 sieve and mixed in a double cone mixer. The mixture was placed in a fluidized bed granulator. Meanwhile, Eudragit RS PO was suspended in purified water to prepare a binding solution. The binding solution was sprayed thereon to form granules, followed by drying. A solution of hydroxypropylmethylcellulose phthalate in a mixture of ethanol and methylene chloride was sprayed to coat the granules.

(2) Preparation of Simvastatin Prior-Release Granules

According to the ingredients and contents illustrated in Table 1 below, simvastatin, microcrystalline cellulose, and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were placed in the high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve, and butylated hydroxyanisole was added thereto, followed by mixing to prepare the title granules.

(3) Mixing and Filling

The final compositions of Processes (1) and (2) were mixed in a double cone mixer. The mixture was mixed with sodium starch glycolate given in a prior-release compartment of Table 1, using a double cone mixer, followed by mixing with colloidal silicon dioxide, and final mixing with magnesium stearate. The final mixture was placed in a powder feeder, and filled in two No. 1 gelatin hard capsules using a capsule filling machine to prepare the title capsules each containing both 180 mg of diltiazem and 10 mg of simvastatin.

### Example 8

### Two-phase preparation of diltiazem-simvastatin capsules

(1) Preparation of Diltiazem Delayed-Release Granules

According to the ingredients and contents illustrated in Table 1 below, diltiazem hydrochloride, fumaric acid and hydroxypropylmethylcellulose were sieved through a No. 35 sieve and mixed in a double cone mixer. Meanwhile, Eudragit L100 and ethylcellulose were dissolved in a mixture of ethanol and methylene chloride to prepare a binding solution. The binding solution was sprayed thereon to form granules, followed by drying to prepare the title capsules.

(2) Preparation of Simvastatin Prior-Release Granules

According to the ingredients and contents illustrated in Table 1 below, simvastatin, microcrystalline cellulose, and mannitolwere sieved through a No. 35 sieve and mixed in a high-speed mixer. Meanwhile, hydroxypropylcellulose and citric acid were dissolved in purified water to prepare a binding solution. The binding solution and the mixture of main ingredients were placed in the high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve, and butylated hydroxyanisole was added thereto, followed by mixing to prepare the title prior-release granules.

(3) Mixing and Filling

The final compositions prepared in Processes (1) and (2) were mixed with sodium starch glycolate given in a prior-release compartment of Table 1, using a double cone mixer, followed by mixing with colloidal silicon dioxide, and final mixing with magnesium stearate.

The final composition was filled in two No. 1 hydroxypropylmethylcellulose hard capsules using a capsule filling machine to prepare the title capsules each containing both 180 mg of diltiazem and 10 mg of simvastatin.

### Example 9

### Two-Phase Preparation of Diltiazem-Simvastatin Capsules

(1) Preparation of Diltiazem Delayed-Release Granules

According to the ingredients and contents illustrated in Table 2 below, diltiazem hydrochloride, fumaric acid and polyethylene oxide were sieved through a No. 35 sieve and mixed in a double cone mixer. The mixture was placed in a fluidized bed granulator. Meanwhile, hydroxypropylmethylcellulose was dissolved in purified water to prepare a binding solution. The binding solution was sprayed thereon to form granules, followed by drying. A solution of hydroxypropylmethylcellulose phthalate in a mixture of ethanol and methylene chloride was sprayed and coated on the granules to prepare the title granules.

(2) Preparation of Simvastatin Prior-Release Tablets

According to the ingredients and contents illustrated in Table 2 below, simvastatin, microcrystalline cellulose, and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were placed in the high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve, and butylated hydroxyanisole was added and mixed therewith, followed by mixing with sodium starch glycolate in a double cone mixer, mixing with colloidal silicon dioxide, and final mixing with magnesium stearate.

The final mixture was compressed to prepare the title tablets containing 10 mg of simvastatin per tablet, using a rotary tablet press.

(3) Capsule Filling

The final compositions of Processes (1) and (2) were filled in two No. 1 hydroxypropylmethylcellulose hard capsules using a capsule filling machine to prepare the title capsules each containing both 180 mg of diltiazem and 10 mg of simvastatin.

### Example 10

### Two-Phase Preparation of Diltiazem-Simvastatin Capsules

(1) Preparation of Diltiazem Delayed-Release Granules

According to the ingredients and contents illustrated in Table 2 below, diltiazem hydrochloride, fumaric acid and Carbomer 71G were sieved through a No. 35 sieve and mixed in a double cone mixer. Kollicoat SR30D was sprayed thereon to form granules, followed by drying to prepare the title granules.

(2) Preparation of Simvastatin Prior-Release Tablets

According to the ingredients and contents illustrated in Table 2 below, simvastatin, microcrystalline cellulose, and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were placed in the high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve. Butylated hydroxyanisole was mixed with the sieved material, followed by mixing with sodium starch glycolate in a double cone mixer, mixing with colloidal silicon dioxide, and final mixing with magnesium stearate.

The final mixture was compressed to prepare the title tablets containing 10 mg of simvastatin per tablet, using a rotary tablet press.

(3) Capsule Filling

The final compositions of Processes (1) and (2) were filled in two No. 1 hydroxypropylmethylcellulose hard capsules using a capsule filling machine to prepare the title capsules each containing both 180 mg of diltiazem and 10 mg of simvastatin.

### Example 11

### Preparation of Diltiazem-Lovastatin Multi-Layered Tablets

(1) Preparation of Diltiazem Delayed-Release Granules

According to the ingredients and contents illustrated in Table 2 below, diltiazem hydrochloride, fumaric acid and polyethylene oxide were sieved through a No. 35 sieve and mixed in a double cone mixer. The mixture was placed in a fluidized bed granulator. Meanwhile, hydroxypropylmethylcellulose was dissolved in purified water to prepare a binding solution which was then sprayed thereon to form granules, followed by drying. A solution of Eudragit RS PO dissolved in a mixture of ethanol and methylene chloride was sprayed and coated on the granules to prepare the title granules.

(2) Preparation of Lovastatin Granules

According to the ingredients and contents illustrated in Table 2 below, lovastatin, microcrystalline cellulose, and D-mannitol were sieved through a No. 35 sieve and mixed in a high-speed mixer. Meanwhile, hydroxypropylcellulose and citric acid were dissolved in purified water to prepare a binding solution which was then kneaded with the mixture of main ingredients. After completion of the kneading process, the kneaded material was granulated using an oscillator with a No. 20 sieve, and the granules were dried in a hot-water dryer at 60° C. After completion of the drying process, the granules were sieved again through a No. 20 sieve. Butylated hydroxyanisole was mixed with the sieved material to prepare the title granules.

(3) Post-Mixing, Compression and Coating

The final compositions of Processes (1) and (2) were mixed in a double cone mixer. The mixture was mixed with sodium starch glycolate and colloidal silicon dioxide given in a prior-release compartment of Table 2 and finally mixed with magnesium stearate in a high-speed mixer.

The final mixture was compressed into tablets using a rotary tablet press. Using a coating solution prepared by mixing coating layer ingredients given in Table 2, a film-coating layer was formed on the compressed tablets by a Hi-coater, thus preparing the title two-phase matrix tablets.

### Example 12

### Preparation of Diltiazem-Atorvastatin Multi-Layered Tablets

(1) Preparation of Diltiazem Delayed-Release Layer

According to the ingredients and contents illustrated in Table 2 below, diltiazem hydrochloride, fumaric acid and Carbomer 71G were sieved through a No. 35 sieve, mixed in a double cone mixer and then placed in a high-speed mixer, followed by kneading with Kollicoat SR30D. After completion of the kneading process, the kneaded material was granulated using an oscillator with a No. 20 sieve, and the granules were dried in a hot-water dryer at 60° C. After completion of the drying process, the granules were sieved again through a No. 20 sieve. Magnesium stearate was added thereto, followed by final mixing in a double cone mixer to prepare the title delayed-release layer.

(2) Preparation of Atorvastatin Prior-Release Layer

According to the ingredients and contents illustrated in Table 2 below, atorvastatin calcium, microcrystalline cellulose, D-mannitol, and calcium carbonate were sieved through a No. 35 sieve and mixed in a high-speed mixer. Meanwhile, hydroxypropylcellulose and citric acid were dissolved in purified water to prepare a binding solution. The binding solution and the mixture of main ingredients were placed in a high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve. The sieved material was mixed with sodium starch glycolate and colloidal silicon dioxide, and magnesium stearate was added thereto, followed by final mixing in a double cone mixer to prepare the title prior-release layer.

(3) Compression and Coating

In a multilayer tablet press, the final atorvastatin-containing composition of Process (2) was placed in a first powder feeder, and the final diltiazem-containing composition of Process (1) was placed in a second powder feeder. The compositions in the feeders were compressed into tablets under such conditions that the interlayer incorporation may be minimized Using a coating solution prepared by mixing coating layer ingredients given in Table 2, a film-coating layer was formed on the compressed tablets by a Hi-coater, thus preparing the title multi-layered tablets.

### Example 13

### Two-Phase Preparation of Diltiazem-Lovastatin Capsules

(1) Preparation of Diltiazem Delayed-Release Pellets

According to the ingredients and contents illustrated in Table 2 below, diltiazem hydrochloride, fumaric acid, and Carbomer 71G were sieved through a No. 35 sieve, and were placed together with sugar seeds in a fluidized bed granulator. Meanwhile, hydroxypropylmethylcellulose was dissolved in purified water to prepare a binding solution. The binding solution was sprayed thereon to form diltiazem-containing pellets, followed by drying. A solution of Eudragit RS PO in a mixture of ethanol and methylene chloride was sprayed and coated on the pellets to prepare the title pellets.

(2) Preparation of Lovastatin Prior-Release Granules

According to the ingredients and contents illustrated in Table 2 below, lovastatin, microcrystalline cellulose, and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were kneaded. After completion of the kneading process, the kneaded material was granulated using an oscillator with a No. 20 sieve, and the granules were dried in a hot-water dryer at 60° C. After completion of the drying process, the granules were sieved again through a No. 20 sieve, and butylated hydroxyanisole was added thereto, followed by mixing to prepare the title granules.

(3) Mixing and Capsule Filling

The final compositions of Processes (1) and (2) were mixed in a double cone mixer. The mixture was mixed with sodium starch glycolate given in a prior-release compartment of Table 2, using a double cone mixer, followed by mixing with colloidal silicon dioxide, and final mixing with magnesium stearate. The final mixture was placed in a powder feeder, and filled in two No. 1 gelatin hard capsules using a capsule filling machine to prepare the title capsules each containing both 180 mg of diltiazem and 10 mg of lovastatin.

### Example 14

### Two-phase preparation of diltiazem-atorvastatin capsules

(1) Preparation of Diltiazem Delayed-Release Pellets

According to the ingredients and contents illustrated in Table 2 below, diltiazem hydrochloride, fumaric acid, and carbomer were sieved through a No. 35 sieve, and were placed together with sugar seeds in a fluidized bed granulator. Meanwhile, hydroxypropylmethylcellulose was dissolved in purified water to prepare a binding solution. The binding solution was sprayed thereon to form diltiazem-containing pellets, followed by drying. Kollicoat SR30D was sprayed on the granules to form pellets which were then dried to prepare the title pellets.

(2) Preparation of Atorvastatin Prior-Release Tablets

According to the ingredients and contents illustrated in Table 2 below, atorvastatin calcium, microcrystalline cellulose, D-mannitol, and calcium carbonate were sieved through a No. 35 sieve and mixed in a high-speed mixer. Meanwhile, hydroxypropylcellulose and citric acid were dissolved in purified water to prepare a binding solution. The binding solution and the mixture of main ingredients were placed and kneaded in a high-speed mixer. After completion of the kneading process, the kneaded material was granulated using an oscillator with a No. 20 sieve, and the granules were dried in a hot-water dryer at 60° C. After completion of the drying process, the granules were sieved again through a No. 20 sieve. Sodium starch glycolate was added to the final composition, followed by mixing in a double cone mixer, mixing with colloidal silicon dioxide, and final mixing with magnesium stearate.

The final mixture was compressed to prepare the title tablets containing 10.85 mg of atorvastatin calcium per tablet, using a rotary tablet press.

(3) Capsule Filling

The final compositions of Processes (1) and (2) were filled in two No. 1 hydroxypropylmethylcellulose hard capsules using a capsule filling machine to prepare the title capsules each containing both 180 mg of diltiazem and 10.85 mg of atorvastatin calcium.

### Example 15

### Two-Phase Preparation of Diltiazem-Atorvastatin Capsules

(1) Preparation of Diltiazem Delayed-Release Granules According to the ingredients and contents illustrated in Table 2 below, diltiazem hydrochloride, fumaric acid and polyethylene oxide were sieved through a No. 35 sieve and mixed in a double cone mixer. The mixture was placed in a fluidized bed granulator. Meanwhile, hydroxypropylmethylcellulose was dissolved in 200 mg of purified water to prepare a binding solution. The binding solution was sprayed thereon to form granules, followed by drying. A binding solution of Eudragit RS PO suspended in purified water was sprayed and coated on the granules to prepare the title granules.

(2) Preparation of Atorvastatin Prior-Release Granules

According to the ingredients and contents illustrated in Table 2 below, atorvastatin calcium, microcrystalline cellulose, calcium carbonate, and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were placed and kneaded in a high-speed mixer. After completion of the kneading process, the kneaded material was granulated using an oscillator with a No. 20 sieve, and the granules were dried in a hot-water dryer at 60° C. After completion of the drying process, the granules were sieved again through a No. 20 sieve to prepare the title granules.

(3) Mixing and Filling

The final compositions of Processes (1) and (2) were mixed in a double cone mixer.

Sodium starch glycolate given in a prior-release compartment was added thereto, followed by mixing in a double cone mixer, mixing with colloidal silicon dioxide, and final mixing with magnesium stearate. The final mixture was placed in a powder feeder, and filled in two No. 1 gelatin hard capsules using a capsule filling machine to prepare the title capsules each containing both 180 mg of diltiazem and 10.85 mg of atorvastatin calcium.

### Example 16

### Two Phase Preparation of Diltiazem-Lovastatin Capsules

(1) Preparation of Diltiazem Delayed-Release Granules

According to the ingredients and contents illustrated in Table 2 below, diltiazem hydrochloride, fumaric acid, polyethylene oxide and Carbomer 71G were sieved through a No. 35 sieve and mixed in a double cone mixer. Kollicoat SR30D was sprayed thereon to form granules, followed by drying to prepare the title granules.

(2) Preparation of Lovastatin Prior-Release Tablets

According to the ingredients and contents illustrated in Table 2 below, lovastatin, microcrystalline cellulose, and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were placed in a high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve. Butylated hydroxyanisole was added thereto, followed by mixing.

Sodium starch glycolate was added to the final composition, followed by mixing in a double cone mixer, mixing with colloidal silicon dioxide, and final mixing with magnesium stearate.

The final mixture was compressed to prepare the title tablets containing 20 mg of lovastatin per tablet, using a rotary tablet press.

(3) Capsule Filling

The final compositions of Processes (1) and (2) were filled in two No. 1 gelatin hard capsules using a capsule filling machine to prepare the title capsules each containing both 180 mg of diltiazem and 10 mg of lovastatin.

### Example 17

### Two-Phase Preparation of Diltiazem-Rosuvastatin Capsules

(1) Preparation of Diltiazem Delayed-Release Granules

According to the ingredients and contents illustrated in Table 3 below, diltiazem hydrochloride, fumaric acid and polyethylene oxide were sieved through a No. 35 sieve and mixed in a double cone mixer. The mixture was placed in a fluidized bed granulator. Meanwhile, polyvinylpyrrolidone was dissolved in purified water to prepare a binding solution. The binding solution was sprayed thereon to form granules, followed by drying. A solution of hydroxypropylmethylcellulose phthalate dissolved in a mixture of ethanol and methylene chloride was sprayed and coated on the granules to prepare the title granules.

(2) Preparation of Rosuvastatin Prior-Release Tablets

According to the ingredients and contents illustrated in Table 3 below, rosuvastatin calcium, microcrystalline cellulose, and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were placed and kneaded in a high-speed mixer. After completion of the kneading process, the kneaded material was granulated using an oscillator with a No. 20 sieve, and the granules were dried in a hot-water dryer at 60° C. After completion of the drying process, the granules were sieved again through a No. 20 sieve.

Sodium starch glycolate was added to the resulting final composition, followed by mixing in a double cone mixer, mixing with colloidal silicon dioxide, and final mixing with magnesium stearate.

The final mixture was compressed to prepare the title tablets containing 20 mg of rosuvastatin per tablet, using a rotary tablet press.

(3) Capsule Filling

The final compositions of Processes (1) and (2) were filled in two No. 1 hydroxypropylmethylcellulose hard capsules using a capsule filling machine to prepare the title capsules each containing both 180 mg of diltiazem and 20.8 mg of rosuvastatin.

### Example 18

### Two-Phase Preparation of Diltiazem-Pitavastatin Capsules

(1) Preparation of Diltiazem Delayed-Release Pellets

According to the ingredients and contents illustrated in Table 3 below, diltiazem hydrochloride, fumaric acid, and hydroxypropylmethylcellulose were sieved through a No. 35 sieve and were placed together with sugar seeds (sugar spheres) in a fluidized bed granulator (GPCG 1: Glatt). Meanwhile, polyvinylpyrrolidone was dissolved in purified water to prepare a binding solution. The binding solution was sprayed thereon to form diltiazem-containing pellets, followed by drying. A solution of hydroxypropylmethylcellulose phthalate dissolved in a mixture of ethanol and methylene chloride was sprayed and coated on the granules to prepare the title pellets.

(2) Preparation of Pitavastatin Granules

According to the ingredients and contents illustrated in Table 3 below, pitavastatin calcium, microcrystalline cellulose, and aluminum magnesium silicate were sieved through a No. 35 sieve and mixed in a high-speed mixer. Meanwhile, hydroxypropylcellulose and citric acid were dissolved in purified water to prepare a binding solution which was then kneaded with the mixture of main ingredients. After completion of the kneading process, the kneaded material was granulated using an oscillator with a No. 20 sieve, and the granules were dried in a hot-water dryer at 60° C. After completion of the drying process, the granules were sieved again through a No. 20 sieve to prepare the title granules.

(3) Mixing and Capsule Filling

The final compositions of Processes (1) and (2) were mixed in a double cone mixer. The mixture was mixed with sodium starch glycolate given in a prior-release compartment, using a double cone mixer, followed by mixing with colloidal silicon dioxide, and final mixing with magnesium stearate. The final mixture was placed in a powder feeder, and filled in two No. 2 gelatin hard capsules using a capsule filling machine to prepare the title capsules each containing both 180 mg of diltiazem and 1 mg of pitavastatin.

### Example 19

### Two-Phase Preparation of Diltiazem-Fluvastatin Capsules

(1) Preparation of Diltiazem Delayed-Release Pellets

According to the ingredients and contents illustrated in Table 3 below, diltiazem hydrochloride, fumaric acid, and polyethylene oxide were sieved through a No. 35 sieve and were placed together with sugar seeds (sugar spheres) in a fluidized bed granulator (GPCG 1: Glatt). Meanwhile, hydroxypropylmethylcellulose was dissolved in purified water to prepare a binding solution. The binding solution was sprayed thereon to form diltiazem-containing pellets, followed by drying. Kollicoat SR30D was sprayed on the granules to form pellets, followed by drying to prepare the title pellets.

(2) Preparation of Fluvastatin Prior-Release Tablets

According to the ingredients and contents illustrated in Table 3 below, fluvastatin sodium, microcrystalline cellulose, D-mannitol, and potassium carbonate were sieved through a No. 35 sieve and mixed in a high-speed mixer. Meanwhile, hydroxypropylcellulose was dissolved in purified water to prepare a binding solution. The binding solution and the mixture of main ingredients were placed in a high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve.

Sodium starch glycolate was added to the final composition, followed by mixing in a double cone mixer, followed by mixing with colloidal silicon dioxide, and final mixing with magnesium stearate.

The final mixture was compressed to prepare the title tablets containing 20 mg of fluvastatin per tablet, using a rotary tablet press.

(3) Capsule Filling

The final compositions of Processes (1) and (2) were filled in two No. 1 hydroxypropylmethylcellulose hard capsules using a capsule filling machine to prepare the title capsules each containing both 180 mg of diltiazem and 20 mg of fluvastatin.

### Example 20

### Preparation of Verapamil-Rosuvastatin Two-Phase Matrix Tablets

(1) Preparation of Verapamil Delayed-Release Granules

According to the ingredients and contents illustrated in Table 3 below, verapamil hydrochloride and hydroxypropylmethylcellulose were sieved through a No. 35 sieve and mixed. The mixture was placed in a high-speed mixer, and a binding solution of Eudragit RS PO dissolved in a mixture of ethanol and methylene chloride was added thereto, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve to prepare the title tablets.

(2) Preparation of Rosuvastatin Prior-Release Granules

According to the ingredients and contents illustrated in Table 3 below, rosuvastatin calcium, microcrystalline cellulose, and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were placed in the high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve to prepare the title granules.

(3) Post-Mixing, Compression and Coating

The final compositions of Processes (1) and (2) were mixed in a double cone mixer, followed by mixing with sodium starch glycolate and colloidal silicon dioxide given in a prior-release compartment of Table 3, and final mixing with magnesium stearate using the double cone mixer.

The final mixture was compressed into tablets, using a rotary tablet press. Using a coating solution prepared by mixing coating layer ingredients given in Table 3, a film-coating layer was formed on the compressed tablets by a Hi-coater, thus preparing the title two-phase matrix tablets.

### Example 21

### Two-Phase Preparation of Verapamil-Pitavastatin Capsules

(1) Preparation of Verapamil Delayed-Release Granules

According to the ingredients and contents illustrated in Table 3 below, verapamil hydrochloride and hydroxypropylmethylcellulose were sieved through a No. 35 sieve and mixed in a double cone mixer. The mixture was placed in a fluidized bed granulator. Meanwhile, a binding solution of Eudragit RS PO suspended in purified water was sprayed thereon to form granules, followed by drying. A solution of hydroxypropylmethylcellulose phthalate dissolved in a 1:1 mixture of ethanol and methylene chloride was sprayed and coated on the granules to prepare the title granules.

(2) Preparation of Pitavastatin Prior-Release Granules

According to the ingredients and contents illustrated in Table 3 below, pitavastatin calcium, microcrystalline cellulose and aluminum magnesium silicate were sieved through a No. 35 sieve and mixed in a high-speed mixer. Meanwhile, hydroxypropylcellulose and citric acid were dissolved in purified water to prepare a binding solution. The binding solution and the mixture of main ingredients were placed in a high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve to prepare the title granules.

(3) Mixing and Filling

The final compositions of Processes (1) and (2) were mixed in a double cone mixer, followed by mixing with sodium starch glycolate given in a prior-release compartment of Table 3, mixing with colloidal silicon dioxide and final mixing with magnesium stearate. The final mixture was placed in a powder feeder, and filled in two No. 2 gelatin hard capsules using a capsule filling machine to prepare the title capsules each containing both 120 mg of verapamil and 1 mg of pitavastatin.

### Example 22

### Preparation of Verapamil-Fluvastatin Multi-Layered Tablets

(1) Preparation of Verapamil Delayed-Release Layer

According to the ingredients and contents illustrated in Table 3 below, verapamil hydrochloride and polyethylene oxide were sieved through a No. 35 sieve and mixed in a double cone mixer. Meanwhile, ethylcellulose was dissolved in 200 mg of ethanol to prepare a binding solution which was then sprayed thereon to form granules, followed by drying. A solution of hydroxypropylmethylcellulose phthalate dissolved in a 1:1 (300 mg:300 mg) mixture of ethanol and methylene chloride was sprayed and coated on the granules. Magnesium stearate was added thereto, followed by final mixing in a double cone mixer to prepare the title delayed-release layer.

(2) Preparation of Fluvastatin Prior-Release Layer

According to the ingredients and contents illustrated in Table 3 below, fluvastatin sodium, microcrystalline cellulose, D-mannitol and potassium carbonate were sieved through a No. 35 sieve and mixed in a high-speed mixer. Meanwhile, hydroxypropylcellulose was dissolved in purified water to prepare a binding solution. The binding solution and the mixture of main ingredients were placed in a high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve. The sieved material was mixed with sodium starch glycolate and colloidal silicon dioxide, followed by final mixing with magnesium stearate in a double cone mixer to prepare the title prior-release layer.

(3) Compression and Coating

In a multilayer tablet press, the final fluvastatin-containing composition of Process (2) was placed in a first powder feeder, and the final verapamil-containing composition of Process (1) was placed in a second powder feeder. The compositions in the feeders were compressed into tablets under such conditions that the interlayer incorporation may be minimized Using a coating solution prepared by mixing coating layer ingredients given in Table 3, a film-coating layer was formed on the compressed tablets by means of a Hi-coater, thus preparing the title sustained-release tablets in the form of a multi-layered tablet.

### Example 23

### Preparation of Verapamil-Simvastatin Two-Phase Matrix Tablets

(1) Preparation of Verapamil Delayed-Release Granules

According to the ingredients and contents illustrated in Table 4 below, verapamil hydrochloride and hydroxypropylmethylcellulose were sieved through a No. 35 sieve and mixed in a double cone mixer. The mixture was placed in a fluidized bed granulator. Meanwhile, polyvinylpyrrolidone was dissolved in purified water to prepare a binding solution. The binding solution was sprayed thereon to form granules, followed by drying. A binding solution of Eudragit RS PO dissolved in a mixture of ethanol and methylene chloride was sprayed and coated on the granules to prepare the title granules.

(2) Preparation of Simvastatin Prior-Release Granules

According to the ingredients and contents illustrated in Table 4 below, simvastatin, microcrystalline cellulose, and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were kneaded. After completion of the kneading process, the kneaded material was granulated using an oscillator with a No. 20 sieve, and the granules were dried in a hot-water dryer at 60° C. After completion of the drying process, the granules were sieved again through a No. 20 sieve, and butylated hydroxyanisole was added thereto, followed by mixing to prepare the title granules.

(3) Post-Mixing, Compression and Coating

The final compositions of Processes (1) and (2) were mixed in a double cone mixer, followed by mixing with sodium starch glycolate and colloidal silicon dioxide given in a prior-release compartment of Table 4, and final mixing with magnesium stearate by a high-speed mixer.

The final mixture was compressed into tablets, using a rotary tablet press. Using a coating solution prepared by mixing coating layer ingredients given in Table 4, a film-coating layer was formed on the compressed tablets by a Hi-coater, thus preparing the title two-phase matrix tablets.

### Example 24

### Preparation of Verapamil-Lovastatin Multi-Layered Tablets

(1) Preparation of Verapamil Delayed-Release Layer

According to the ingredients and contents illustrated in Table 4 below, verapamil hydrochloride and hydroxypropylmethylcellulose were sieved through a No. 35 sieve and mixed in a double cone mixer. The mixture was placed in a high-speed mixer and Kollicoat SR30D was added thereto, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve. Magnesium stearate was added thereto, followed by final mixing in a double cone mixer to prepare the title delayed-release layer.

(2) Preparation of Lovastatin Prior-Release Layer

According to the ingredients and contents illustrated in Table 4 below, lovastatin, microcrystalline cellulose, and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were placed in a high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve. Butylated hydroxyanisole, sodium starch glycolate, and colloidal silicon dioxide were mixed with the sieved material, and magnesium stearate was added thereto, followed by final mixing in a double cone mixer to prepare the title prior-release layer.

(3) Compression and Coating

In a multilayer tablet press, the final lovastatin-containing composition of Process (2) was placed in a first powder feeder, and the final verapamil-containing composition of Process (1) was placed in a second powder feeder. The compositions in the feeders were compressed into tablets under such conditions that the interlayer incorporation may be minimized Using a coating solution prepared by mixing coating layer ingredients given in Table 4, a film-coating layer was formed on the compressed tablets by a Hi-coater, thus preparing the title sustained-release tablets in the form of a multi-layered tablet.

### Example 25

### Preparation of Verapamil-Pravastatin Capsules

(1) Preparation of Verapamil Delayed-Release Granules

According to the ingredients and contents illustrated in Table 4 below, verapamil hydrochloride and polyethylene oxide were sieved through a No. 35 sieve and mixed in a double cone mixer. The mixture was placed in a fluidized bed granulator. Meanwhile, polyvinylpyrrolidone was dissolved in purified water to prepare a binding solution. The binding solution was sprayed thereon to form granules, followed by drying. A solution of hydroxypropylmethylcellulose phthalate dissolved in a mixture of ethanol and methylene chloride was sprayed and coated on the granules to prepare the title granules.

(2) Preparation of Pravastatin Prior-Release Tablets

According to the ingredients and contents illustrated in Table 4 below, pravastatin sodium, microcrystalline cellulose and D-mannitol were sieved through a No. 35 sieve and mixed in a high-speed mixer. Meanwhile, hydroxypropylcellulose and citric acid were dissolved in water to prepare a binding solution. The binding solution and the mixture of main ingredients were placed in a high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve. Sodium starch glycolate and colloidal silicon dioxide were mixed therewith, followed by final mixing with magnesium stearate in a double cone mixer. Then, the final mixture was compressed into a tablet containing 10 mg of pravastatin using a rotary tablet press.

(3) Capsule Filling

The final compositions of Processes (1) and (2) were filled in two No. 1 gelatin hard capsules using a capsule filling machine to prepare the title capsules each containing both 120 mg of verapamil and 10 mg of pravastatin.

### Example 26

### Two-Phase Preparation of Verapamil-Simvastatin Capsules

(1) Preparation of Verapamil Delayed-Release Granules

According to the ingredients and contents illustrated in Table 4 below, verapamil hydrochloride and Carbomer 71G were sieved through a No. 35 sieve and mixed in a double cone mixer. Then, Kollicoat SR30D was sprayed thereon to form granules, followed by drying to prepare the title granules.

(2) Preparation of Simvastatin Prior-Release Granules

According to the ingredients and contents illustrated in Table 4 below, simvastatin, microcrystalline cellulose, and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were placed in a high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve. Butylated hydroxyanisole was added thereto, followed by mixing to prepare the title granules.

(3) Mixing and Filling

The final compositions of Processes (1) and (2) were mixed in a double cone mixer, followed by mixing with sodium starch glycolate given in a prior-release compartment in a double cone mixer, mixing with colloidal silicon dioxide and final mixing with magnesium stearate. The final composition was filled in two No. 1 gelatin hard capsules using a capsule filling machine to prepare the title capsules each containing both 120 mg of verapamil and 10 mg of simvastatin.

### Example 27

### Two-Phase Preparation of Verapamil-Lovastatin Capsules

(1) Preparation of Verapamil Delayed-Release Pellets

According to the ingredients and contents illustrated in Table 4 below, verapamil hydrochloride and hydroxypropylmethylcellulose were sieved through a No. 35 sieve and were placed together with sugar seeds in a fluidized bed granulator. Meanwhile, polyvinylpyrrolidone was dissolved in purified water to prepare a binding solution. The binding solution was sprayed thereon to form verapamil-containing pellets, followed by drying. A solution of hydroxypropylmethylcellulose phthalate dissolved in a mixture of ethanol and methylene chloride was sprayed on the pellets to form pellets, followed by drying to prepare the title pellets.

(2) Preparation of Lovastatin Prior-Release Tablets

According to the ingredients and contents illustrated in Table 4 below, lovastatin, microcrystalline cellulose and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were placed in a high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve. Butylated hydroxyanisole was added thereto, followed by mixing.

Sodium starch glycolate was added to the resulting final composition, followed by mixing in a double cone mixer, mixing with colloidal silicon dioxide, and final mixing with magnesium stearate.

The final mixture was compressed to prepare the title tablets containing 10 mg of lovastatin per tablet, using a rotary tablet press.

(3) Capsule Filling

The final compositions of Processes (1) and (2) were filled in two No. 1 hydroxypropylmethylcellulose hard capsules using a capsule filling machine to prepare the title capsules each containing both 120 mg of verapamil and 10 mg of lovastatin.

### Example 28

### Two-Phase Preparation of Verapamil-Pravastatin Capsules

(1) Preparation of Verapamil Delayed-Release Granules

According to the ingredients and contents illustrated in Table 4 below, verapamil hydrochloride and hydroxypropylmethylcellulose were sieved through a No. 35 sieve and mixed in a double cone mixer. Kollicoat SR30D was sprayed thereon to form granules, followed by drying to prepare the title granules.

(2) Preparation of Pravastatin Prior-Release Tablets

According to the ingredients and contents illustrated in Table 4 below, pravastatin sodium, microcrystalline cellulose and D-mannitol were sieved through a No. 35 sieve and mixed in a high-speed mixer. Meanwhile, hydroxypropylcellulose and citric acid were dissolved in purified water to prepare a binding solution. The binding solution and the mixture of main ingredients were placed in a high-speed mixer, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve.

Sodium starch glycolate was added to the final composition, followed by mixing in a double cone mixer, further mixing with colloidal silicon dioxide, and final mixing with magnesium stearate.

The final mixture was compressed to prepare the title tablets containing 20 mg of pravastatin per tablet, using a rotary tablet press.

(3) Capsule Filling

The final compositions of Processes (1) and (2) were filled in two No. 1 gelatin hard capsules using a capsule filling machine to prepare the title capsules each containing both 240 mg of verapamil and 20 mg of pravastatin.

### Example 29

### Diltiazem-Simvastatin Blister Package Kits

Each of Diltiazem delayed-release granules (Process 1) of Example 3 and simvastatin prior-release granules (Process 2) of Example 3 was separately compressed into a tablet using a rotary tablet press and then the tablet including diltiazem and the tablet including simvastatin were packed together in a blister package container (silver foil, Dong-il Corporation, PVDC, Jeon Min Industry Co., Ltd., South Korea) such that they may be simultaneously administered, using a blister package machine (Minister A, Heung—A Engineering, South Korea).

TABLE 1

| | | Composition ratio (mg/tablet) | | | | | | | |
|---|---|---|---|---|---|---|---|---|---|
| | | Example No. | | | | | | | |
| Ingredients | | 1 | 2 | 3 | 4 | 5 | 6 | 7 | 8 |
| Delayed-release | Diltiazem | 360 | 360 | 360 | 360 | 360 | 360 | 360 | 360 |
| compartment | hydrochloride | | | | | | | | |
| | Fumaric acid | 90 | 90 | 90 | 90 | 75 | 75 | 90 | 90 |
| | Sugar seed | — | — | — | — | 72.5 | 72.5 | — | — |
| | Hydroxypropyl | 71 | 71 | 71 | 71 | 52.5 | 52.5 | 70 | 70 |
| | methylcellulose | | | | | | | | |
| | Kollicoat | 110 | | — | — | — | 90 | — | — |
| | SR30D¹) | | | | | | | | |
| | Eudragit RS | — | 90 | — | — | — | — | 90 | — |
| | PO2) | | | | | | | | |
| | Eudragit L100³⁾ | — | — | — | — | — | — | — | 100 |
| | Ethylcellulose | — | 20 | 20 | 20 | — | — | — | 20 |
| | Hydroxypropyl | — | — | 90 | 90 | 90 | — | 30 | — |
| | methylcellulose | | | | | | | | |
| | phthalate | | | | | | | | |
| | Magnesium | — | — | 5 | 5 | — | — | — | — |
| | stearate | | | | | | | | |
| | Purified water | | | | | 550 | 550 | 900 | |
| | Ethanol | | 200/450 | 200/450 | 200/450 | 450 | | 150 | 600 |
| | Methylene | | 450 | 450 | 450 | 450 | | 150 | 600 |
| | chloride | | | | | | | | |
| Prior-release | Simvastatin | 20 | 20 | 20 | 20 | 20 | 20 | 20 | 20 |
| compartment | Lovastatin | — | — | — | — | — | — | — | — |
| | Atorvastatin | — | — | — | — | — | — | — | — |
| | Macrocrystalline | 57 | 57 | 57 | 57 | 57 | 57 | 57 | 57 |
| | cellulose | | | | | | | | |
| | D-mannitol | 112.46 | 112.46 | 112.46 | 112.46 | 112.46 | 112.46 | 112.46 | 112.46 |
| | Sodium starch | 1 | 1 | 1 | 1 | 1 | 1 | 1 | 1 |
| | glycolate | | | | | | | | |
| | Butylated | 0.04 | 0.04 | 0.04 | 0.04 | 0.04 | 0.04 | 0.04 | 0.04 |
| | hydroxyanisole | | | | | | | | |
| | Hydroxypropyl | 5 | 5 | 5 | 5 | 5 | 5 | 5 | 5 |
| | cellulose | | | | | | | | |
| | Colloidal | 1 | 1 | 1 | 1 | 1 | 1 | 1 | 1 |
| | silicon dioxide⁴⁾ | | | | | | | | |
| | Citric acid | 2 | 2 | 2 | 2 | 2 | 2 | 2 | 2 |
| | Magnesium | 1.5 | 1.5 | 1.5 | 1.5 | 1.5 | 1.5 | 1.5 | 1.5 |
| | stearate | | | | | | | | |
| | Purified water | 55 | 55 | 55 | 55 | 55 | 55 | 55 | 55 |
| Coating | Hydroxypropyl | 2.6 | 2.6 | 2.6 | 2.6 | | | | |
| layer | methylcellulose | | | | | | | | |
| | 2910 | | | | | | | | |
| | Hydroxypropyl | 2.6 | 2.6 | 2.6 | 2.6 | | | | |
| | cellulose | | | | | | | | |
| | Titanium oxide | 2.3 | 2.3 | 2.3 | 2.3 | | | | |
| | Talc | 1.5 | 1.5 | 1.5 | 1.5 | | | | |
| | Ethanol | 255 | 255 | 255 | 255 | | | | |
| | Purified water | 63.75 | 63.75 | 63.75 | 63.75 | | | | |

| | | | | | | | | |
|---|---|---|---|---|---|---|---|---|
| Total | 856 | 856 | 861 | 861 | 850 | 850 | 840 | 840 |

¹⁾Kollicoat SR30D - Main ingredient: polyvinyl acetate 30% suspension (BASF)
²⁾Eudragit RS PO - Main ingredient: polyammoniomethacrylate copolymer (Degussa)
³⁾Eudragit RL100 - Main ingredient: polymethacrylic acid copolymer (Degussa)
⁴⁾Colloidal silicon dioxide - trade name: Aerosil 200 (Degussa)

TABLE 2

| | | Composition ratio (mg/tablet) | | | | | | | |
|---|---|---|---|---|---|---|---|---|---|
| | | Example No. | | | | | | | |
| Ingredients | | 9 | 10 | 11 | 12 | 13 | 14 | 15 | 16 |
| Delayed-release | Diltiazem | 360 | 360 | 360 | 360 | 360 | 360 | 360 | 360 |
| compartment | hydrochloride | | | | | | | | |
| | Fumaric acid | 90 | 90 | 90 | 90 | 75 | 75 | 90 | 90 |
| | Sugar seed | — | — | — | — | 72.5 | 72.5 | — | — |
| | Polyethylene oxide | 71 | — | 71 | — | — | — | 71 | 72.83 |
| | Kollicoat SR30D¹) | — | 90 | — | 90 | — | 90 | — | 90 |
| | Eudragit RS PO²⁾ | — | — | 90 | — | 90 | — | 90 | — |
| | Carbomer 71G³⁾ | — | 91 | — | 91 | 20 | 22.5 | — | 91 |
| | Hydroxypropyl | 20 | — | 20 | — | 22.5 | 22.5 | 20 | — |
| | methylcellulose | | | | | | | | |
| | Ethylcellulose | — | 20 | 20 | 20 | — | — | — | 20 |
| | Hydroxypropyl | 90 | — | — | — | — | — | — | — |
| | methylcellulose | | | | | | | | |
| | phthalate | | | | | | | | |
| | Magnesium stearate | — | — | — | 5 | — | — | — | — |
| | Purified water | 200 | | 200 | | 250 | 250 | 200/900 | |
| | Ethanol | 450 | | 450 | | 450 | | | |
| | Methylene chloride | 450 | | 450 | | 450 | | | |
| Prior-release | Simvastatin | 20 | 20 | — | — | — | — | — | — |
| compartment | Lovastatin | — | — | 20 | — | 20 | — | — | 20 |
| | Atorvastatin calcium | — | — | — | 21.7 | — | 21.7 | 21.7 | — |
| | Macrocrystalline | 57 | 57 | 57 | 57 | 57 | 57 | 57 | 57 |
| | cellulose | | | | | | | | |
| | Calcium carbonate | — | — | — | 20 | — | 20 | 20 | — |
| | D-mannitol | 112.46 | 112.46 | 112.46 | 92.8 | 112.46 | 92.8 | 92.8 | 112.46 |
| | Sodium starch | 1 | 1 | 1 | 1 | 1 | 1 | 1 | 1 |
| | glycolate | | | | | | | | |
| | Butylated | 0.04 | 0.04 | 0.04 | — | 0.04 | — | — | 0.04 |
| | hydroxyanisole | | | | | | | | |
| | Hydroxypropyl | 5 | 5 | 5 | 5 | 5 | 5 | 5 | 5 |
| | cellulose | | | | | | | | |
| | Colloidal silicon | 1 | 1 | 1 | 1 | 1 | 1 | 1 | 1 |
| | dioxide⁴⁾ | | | | | | | | |
| | Citric acid | 2 | 2 | 2 | 2 | 2 | 2 | 2 | 2 |
| | Magnesium stearate | 1.5 | 1.5 | 1.5 | 1.5 | 1.5 | 1.5 | 1.5 | 1.5 |
| | Purified water | 55 | 55 | 55 | 55 | 55 | 55 | 55 | 55 |
| Coating | Hydroxypropyl | — | — | 7.5 | 7.5 | — | — | — | — |
| layer | methylcellulose 2910 | | | | | | | | |
| | Hydroxypropyl | — | — | 7.5 | 7.5 | — | — | — | — |
| | cellulose | | | | | | | | |
| | Titanium oxide | — | — | 7 | 7 | — | — | — | — |
| | Talc | — | — | 3 | 3 | — | — | — | — |
| | Ethanol | — | — | 255 | 255 | — | — | — | — |
| | Purified water | — | — | 63.75 | 63.75 | — | — | — | — |

| | | | | | | | | |
|---|---|---|---|---|---|---|---|---|
| Total | 831 | 831 | 863 | 863 | 840 | 842 | 833 | 831 |

¹⁾Kollicoat SR30D - Main ingredient: polyvinyl acetate 30% suspension (BASF)
²⁾Eudragit RS PO - Main ingredient: polyammoniomethacrylate copolymer (Degussa)
³⁾Carbomer 71G - Main ingredient: Carboxyvinyl polymer (Lubrizol)
⁴⁾Colloidal silicon dioxide - trade name: Aerosil 200 (Degussa)

TABLE 3

| | | Composition ratio (mg/tablet) | | | | | |
|---|---|---|---|---|---|---|---|
| | | Example No. | | | | | |
| Ingredients | | 17 | 18 | 19 | 20 | 21 | 22 |
| Delayed-release | Diltiazem hydrochloride | 360 | 360 | 360 | — | — | — |
| compartment | Verapamil hydrochloride | — | — | — | 240 | 240 | 240 |
| | Fumaric acid | 90 | 75 | 75 | — | — | — |
| | Hydroxypropylmethylcellulose | — | 52.5 | 20 | 120 | 120 | — |
| | Polyethylene oxide | 71 | — | 52.5 | — | — | 120 |
| | Sugar seed | — | 72.5 | 72.5 | — | — | — |
| | Kollicoat SR30D¹⁾ | — | — | 90 | — | — | — |
| | Eudragit RS PO²⁾ | — | — | — | 60 | 60 | — |
| | Ethylcellulose | — | — | — | — | — | 20 |
| | Polyvinylpyrrolidone | 20 | 20 | — | — | — | — |
| | Hydroxypropylmethylcellulose | 110 | 90 | — | — | 40 | 60 |
| | phthalate | | | | | | |
| | Magnesium stearate | — | — | — | — | — | 3 |
| | Purified water | 200 | 200 | 200 | | 600 | |
| | Ethanol | 550 | 450 | | 300 | 200 | 200/300 |
| | Methylene chloride | 550 | 450 | | 300 | 200 | 300 |
| Prior-release | Rosuvastatin calcium | 41.6 | — | — | 41.6 | — | — |
| compartment | Pitavastatin calcium | — | 2 | — | — | 2 | — |
| | Fluvastatin sodium | — | — | 42.2 | — | — | 42.2 |
| | Microcrystalline cellulose | 57 | 40 | 40.5 | 57 | 40 | 40.5 |
| | D-mannitol | 64.9 | — | 37.8 | 64.9 | — | 38.8 |
| | Calcium carbonate | — | — | 4 | — | — | 4 |
| | Sodium starch glycolate | 1 | 1 | 1 | 1 | 1 | 1 |
| | Aluminum magnesium silicate | | 0.5 | — | — | 0.5 | — |
| | Hydroxypropylcellulose | 5 | 5 | 5 | 5 | 5 | 5 |
| | Colloidal silicon dioxide³⁾ | 1 | 1 | 1 | 1 | 1 | 1 |
| | Citric acid | 2 | 2 | — | 2 | 2 | — |
| | Magnesium stearate | 1.5 | 1.5 | 1.5 | 1.5 | 1.5 | 1.5 |
| | Purified water | 55 | 55 | 55 | 55 | 55 | 55 |
| Coating | Hydroxypropylmethylcellulose | | — | — | 5.2 | — | 5.2 |
| layer | 2910 | | | | | | |
| | Hydroxypropylcellulose | | — | — | 5.2 | — | 5.2 |
| | Titanium oxide | | — | — | 4.6 | — | 4.6 |
| | Talc | | — | — | 3 | — | 3 |
| | Ethanol | | — | — | 129.6 | — | 129.6 |
| | Purified water | | — | — | 32.4 | — | 32.4 |

| | | | | | | |
|---|---|---|---|---|---|---|
| Total | 825 | 723 | 805 | 610 | 511 | 595 |

¹⁾Kollicoat SR30D - Main ingredient: polyvinyl acetate 30% suspension (BASF)
²⁾Eudragit RS PO - Main ingredient: polyammoniomethacrylate copolymer (Degussa)
³⁾Colloidal silicon dioxide - trade name: Aerosil 200 (Degussa)

TABLE 4

| | | Composition ratio (mg/tablet) | | | | | |
|---|---|---|---|---|---|---|---|
| | | Example No. | | | | | |
| Ingredients | | 23 | 24 | 25 | 26 | 27 | 28 |
| | Verapamil hydrochloride | 240 | 240 | 240 | 240 | 240 | 240 |
| | Hydroxypropylmethylcellulose | 120 | 120 | — | — | 75 | 120 |
| | Polyethylene oxide | — | — | 12 | — | — | — |
| | Sugar seed | — | — | — | — | 52 | — |
| | Kollicoat SR30D¹⁾ | — | 60 | — | 60 | — | 60 |
| | Eudragit RS PO²⁾ | 60 | — | — | — | — | — |
| | Carbomer 71G³⁾ | — | — | — | 120 | — | — |
| | Polyvinylpyrrolidone | 16 | — | 16 | — | 16 | — |
| | Hydroxypropylmethylcellulose | — | — | 60 | — | 60 | — |
| | phthalate | | | | | | |
| | Magnesium stearate | — | 4 | — | — | — | — |
| | Purified water | 160 | | 160 | | 160 | |
| | Ethanol | 300 | | 300 | | 300 | |
| | Methylene chloride | 300 | | 300 | | 300 | |
| Prior-release | Simvastatin | 20 | — | — | 20 | — | — |
| compartment | Lovastatin | — | 20 | — | — | 20 | — |
| | Pravastatin sodium | — | — | 20 | — | — | 20 |
| | Microcrystalline cellulose | 57 | 57 | 57 | 57 | 57 | 57 |
| | D-mannitol | 112.96 | 112.96 | 112.5 | 112.46 | 112.46 | 112.5 |
| | Sodium starch glycolate | 1 | 1 | 1 | 1 | 1 | 1 |
| | Butylated hydroxyanisole | 0.04 | 0.04 | — | 0.04 | 0.04 | — |
| | Hydroxypropylcellulose | 5 | 5 | 5 | 5 | 5 | 5 |
| | Colloidal silicon dioxide⁴⁾ | 1 | 1 | 1 | 1 | 1 | 1 |
| | Citric acid | 2 | 2 | 2 | 2 | 2 | 2 |
| | Magnesium stearate | 1.5 | 1.5 | 1.5 | 1.5 | 1.5 | 1.5 |
| | Purified water | 55 | 55 | 55 | 55 | 55 | 55 |
| Coating | Hydroxypropylmethylcellulose | 5.2 | 2.6 | — | — | — | — |
| layer | 2910 | | | | | | |
| | Hydroxypropylcellulose | 5.2 | 2.6 | — | — | — | — |
| | Titanium oxide | 4.6 | 2.3 | — | — | — | — |
| | Talc | 3 | 1.5 | — | — | — | — |
| | Ethanol | 129.6 | 129.6 | — | — | — | — |
| | Purified water | 32.4 | 32.4 | — | — | — | — |

| | | | | | | |
|---|---|---|---|---|---|---|
| Total | 653 | 641 | 636 | 625 | 643 | 620 |

¹⁾Kollicoat SR30D - Main ingredient: polyvinyl acetate 30% suspension (BASF)
²⁾Eudragit RS PO - Main ingredient: polyammoniomethacrylate copolymer (Degussa)
³⁾Carbomer 71G - Main ingredient: Carboxyvinyl polymer (Lubrizol)
⁴⁾Colloidal silicon dioxide - trade name: Aerosil 200 (Degussa)

### Example 30

### Preparation of Diltiazem-Atorvastatin Multi-Layered Tablets

(1) Preparation of Diltiazem Delayed-Release Layer

According to the ingredients and contents illustrated in Table 5 below, diltiazem hydrochloride, microcrystalline cellulose (Avicel PH, FMC Biopolymer, USA), povidone (Kollidon 30, D-BASF, Germany), and fumaric acid (Daejung Chemicals & Metals Co., Ltd., South Korea) were sieved through a No. 35 sieve and mixed in a double cone mixer (Dasan Pharmatech, South Korea). The mixture and purified water (30 mg) were placed and kneaded in a high-speed mixer (Lab. Pharma Mixer P, Diosna, Germany). The kneaded material was extruded through an extruder (EXDCS-100, Fuji Denki Kogyo Company, Japan) and the extrudate was spheronized into an appropriate size. The spheronized material was dried in a hot-water dryer at 60° C. and sieved through a No. 25 sieve to prepare beads. Meanwhile, hydroxypropylmethylcellulose (HYPROMELLOSE, Shin-Etsu Chemical Co., Ltd., Japan), titanium oxide (Kronos, USA), talc (Nippon Talc Co., Ltd., Japan), polysorbate 80 (Duksan Pure Chemical Co., Ltd., South Korea), and simethicone emulsion (polydimethylsiloxane 30%, Dow Corning, USA) were mixed, and a poly(methyl methacrylate ethyl acrylate) copolymer (Eudragit NE 30D, Degussa, Germany) was added thereto to prepare a coating solution. After the preparation of a coating solution was complete, the beads were placed in a fluidized bed coater (GPCG-1, Glatt, Germany) and were coated with the coating solution to an appropriate thickness (about 0.05 mm) The coated beads were dried in an oven at 45° C. After completion of the drying process, a polymethacrylate copolymer (Eudragit L100, Degussa, Germany), a polyvinyl acetate/povidone mixture (Kollidon SR, D-BASF, Germany), and magnesium stearate (N of Corp., Japan) were added thereto, followed by final mixing at room temperature in a double cone mixer (Dasan Pharmatech, South Korea), thereby preparing the title layer.

(2) Preparation of Atorvastatin Prior-Release Layer

According to the ingredients and contents illustrated in Table 5 below, an atorvastatin calcium trihydrate, microcrystalline cellulose, lactose (Lactose 200, DMV Pharm), precipitated calcium carbonate (Nitto Funka Kogyo, Japan), pregelatinized starch (Colorcon, USA), and sodium lauryl sulfate (Duksan Pure Chemical Co., Ltd., South Korea) were sieved through a No. 35 sieve and mixed in a high-speed mixer (Lab. Pharma Mixer P, Diosna, Germany) at room temperature to prepare a mixture of main ingredients. Meanwhile, hydroxypropylcellulose was dissolved in water to prepare a binding solution. The binding solution was added to the high-speed mixer (Lab. Pharma Mixer P, Diosna, Germany) where the mixture of main ingredients is present, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve (KYK-60, KoreaMedi Co., Ltd., South Korea) to prepare granules. The granules were mixed with croscarmellose sodium (Acdisol, DMV Pharm, Germany) and colloidal silicon dioxide (Aerosil 200, Degussa, Germany), and magnesium stearate was added thereto, followed by final mixing in a double cone mixer (Dasan Pharmatech, South Korea) to prepare the title layer.

(3) Compression and Coating

In a multilayer tablet press (MRC-37T, Sejong Pharmatech Co., Ltd., South Korea), the atorvastatin prior-release layer of Process (2) was placed in a first powder feeder, and the diltiazem delayed-release layer of Process (1) was placed in a second powder feeder. The layer compositions in the feeders were compressed into tablets. Meanwhile, hydroxypropylmethylcellulose 2910 (Shin-Etsu Chemical Co., Ltd., Japan) and hydroxypropylcellulose were dissolved in ethanol (255 mg) and purified water (63.75 mg), and titanium oxide and talc were dispersed therein to prepare a coating solution. The compressed tablets were coated with the coating solution in a Hi-coater (SFC-30N, Sejong Pharmatech Co., Ltd., South Korea) to form a film-coating layer, thus preparing multi-layered tablets.

### Example 31

### Preparation of Diltiazem-Atorvastatin Two-Phase Matrix Tablets

(1) Preparation of Diltiazem Delayed-Release Granules

According to the ingredients and contents illustrated in Table 5 below, diltiazem hydrochloride, fumaric acid and hydroxypropylmethylcellulose were sieved through a No. 35 sieve, followed by mixing. The mixture was placed in a high-speed mixer (Lab. Pharma Mixer P, Diosna, Germany) and polyvinyl acetate (Kollicoat SR30D, D-BASF, Germany) was added thereto, followed by kneading. Then, kneaded material was sieved using an oscillator with a No. 20 sieve, and the granules were dried in a hot-water dryer at 60° C. After completion of the drying process, the granules were sieved again through a No. 20 sieve (KYK-60, KoreaMedi Co., Ltd., South Korea) to prepare granules. A solution of hydroxypropylmethylcellulose phthalate dissolved in a 1:1 (200 mg:200 mg) mixture of ethanol and methylene chloride was sprayed and coated on the granules to prepare the title granules.

(2) Preparation of Atorvastatin Prior-Release Granules

According to the ingredients and contents illustrated in Table 5 below, atorvastatin calcium trihydrate, microcrystalline cellulose, lactose, precipitated calcium carbonate, pregelatinized starch, and sodium lauryl sulfate were sieved through a No. 35 sieve and mixed in a high-speed mixer (Lab. Pharma Mixer P, Diosna, Germany). Meanwhile, hydroxypropylcellulose was dissolved in purified water to prepare a binding solution. The binding solution was added to the high-speed mixer (Lab. Pharma Mixer P, Diosna, Germany) where the mixture of main ingredients is present, followed by kneading. After completion of the kneading process, the kneaded material was granulated using an oscillator with a No. 20 sieve, and the granules were dried in a hot-water dryer at 60° C. After completion of the drying process, the granules were sieved again through a No. 20 sieve (KYK-60, KoreaMedi Co., Ltd., South Korea) to prepare the title granules.

(3) Post-Mixing, Compression and Coating

The diltiazem delayed-release granules of Process (1) and the atorvastatin prior-release granules of Process (2) were mixed in a double cone mixer at room temperature. The mixture was mixed with croscarmellose sodium and colloidal silicon dioxide in amounts given in a prior-release compartment for Example 31 of Table 5 below, and magnesium stearate was added thereto, followed by final mixing in a double cone mixer to prepare the final mixture.

The final mixture was compressed into tablets using a rotary tablet press (MRC-30, Sejong Pharmatech Co., Ltd., South Korea). Meanwhile, hydroxypropylmethylcellulose 2910 and hydroxypropylcellulose were dissolved in ethanol (255 mg) and purified water (63.75 mg) and titanium oxide and talc were dispersed therein to prepare a coating solution. The compressed tablets were coated with the coating solution in a Hi-coater (SFC-30N, Sejong Pharmatech Co., Ltd., South Korea) to form a film-coating layer, thus preparing the title tablets.

### Example 32

### Preparation of Diltiazem-Atorvastatin Two-Phase Matrix Tablets

(1) Preparation of Diltiazem Delayed-Release Granules

According to the ingredients and contents illustrated in Table 5 below, diltiazem hydrochloride, fumaric acid and hydroxypropylmethylcellulose were sieved through a No. 35 sieve and mixed in a double cone mixer. The mixture was placed in a fluidized bed granulator (GPCG 1, Glatt, Germany). Meanwhile, ethylcellulose (Aqualon, Hercules, USA) was dissolved in ethanol (50 mg) to prepare a binding solution which was then sprayed thereon to form granules, followed by drying. A solution of a poly(ethyl acrylate, methyl methacrylate, trimethylaminoethyl methacrylate) copolymer (Eudragit RS PO, Degussa, Germany) dissolved in a 1:1 (200 mg:200 mg) mixture of ethanol and methylene chloride was sprayed and coated on the granules to prepare the title granules.

(2) Preparation of Atorvastatin Prior-Release Granules

According to the ingredients and contents illustrated in Table 5 below, the title granules were prepared in the same procedure as in Process (2) of Example 31.

(3) Post-Mixing, Compression and Coating

Using the granules prepared in Processes (1) and (2), the title tablets were prepared in the same procedure as in Process (3) of Example 31.

### Example 33

### Preparation of Diltiazem-Atorvastatin Multi-Layered Tablets

(1) Preparation of Diltiazem Delayed-Release Layer

According to the ingredients and contents illustrated in Table 5 below, diltiazem hydrochloride, fumaric acid and hydroxypropylmethylcellulose were sieved through a No. 35 sieve and mixed in a double cone mixer. The mixture was placed in a fluidized bed granulator (GPCG 1, Glatt, Germany). Ethylcellulose was dissolved in ethanol (50 mg) to prepare a binding solution which was then sprayed thereon to form granules, followed by drying. A solution of hydroxypropylmethylcellulose phthalate dissolved in a 1:1 (200 mg:200 mg) mixture of ethanol and methylene chloride was sprayed and coated on the granules. Magnesium stearate was added thereto, followed by final mixing in a double cone mixer to prepare the title layer.

(2) Preparation of Atorvastatin Prior-Release Layer

According to the ingredients and contents illustrated in Table 5 below, the title layer was prepared in the same procedure as in Process (2) of Example 30.

3) Compression and Coating

Using the layers prepared in Processes (1) and (2), the title tablets were prepared in the same procedure as in Process (3) of Example 30.

### Example 34

### Two-Phase Preparation of Diltiazem-Atorvastatin Capsules

(1) Preparation of Diltiazem Delayed-Release Pellets

According to the ingredients and contents illustrated in Table 5 below, diltiazem hydrochloride, fumaric acid, hydroxypropylmethylcellulose and sugar seeds (sugar spheres) were sieved through a No. 35 sieve and were placed together with sugar seeds (sugar spheres) in a fluidized bed granulator (GPCG 1, Glatt, Germany), followed by mixing. Meanwhile, hydroxypropylmethylcellulose was dissolved in purified water (100 mg) to prepare a binding solution. The binding solution was sprayed thereon to form diltiazem-containing pellets, followed by drying. A polyvinyl acetate 30% suspension (Kollicoat SR 30D, D-BASF, Germany) was sprayed on the pellets which were then dried. A solution of hydroxypropylmethylcellulose phthalate dissolved in a 1:1 (200 mg:200 mg) mixture of ethanol and methylene chloride was sprayed and coated on the pellets to prepare the title pellets.

(2) Preparation of Atorvastatin Prior-Release Granules

According to the ingredients and contents illustrated in Table 5 below, the title granules were prepared in the same procedure as in Process (2) of Example 31.

(3) Mixing and Capsule Filling

The pellet products of Processes (1) and (2) were mixed in a double cone mixer at room temperature, and croscarmellose sodium given in a prior-release compartment for Example 34 of Table 5 was added thereto, followed by mixing. Colloidal silicon dioxide was added thereto, and magnesium stearate was finally added thereto, followed by final mixing. The final mixture was placed in a powder feeder and filled in gelatin hard capsules using a capsule filling machine (SF-40N, Sejong Pharmatech Co., Ltd., South Korea) to prepare the title capsules each containing both 360 mg of diltiazem and 10.85 mg of atorvastatin calcium trihydrate.

### Example 35

### Two-Phase Preparation of Diltiazem-Atorvastatin Capsules

(1) Preparation of Diltiazem Delayed-Release Pellets

According to the ingredients and contents illustrated in Table 5 below, diltiazem hydrochloride, fumaric acid, and hydroxypropylmethylcellulose were sieved through a No. 35 sieve and were placed together with sugar seeds (sugar spheres) in a fluidized bed granulator (GPCG 1, Glatt, Germany), followed by mixing. Meanwhile, hydroxypropylmethylcellulose was dissolved in purified water (100 mg) to prepare a binding solution. The binding solution was sprayed thereon to form diltiazem-containing pellets, followed by drying. A polyvinyl acetate 30% suspension (Kollicoat SR 30D, D-BASF, Germany) was sprayed on the diltiazem-containing pellets which were then dried. A solution of polymethacrylate copolymer (Eudragit L100, D-BASF, Germany) dissolved in a 1:1 (200 mg:200 mg) mixture of ethanol and methylene chloride was sprayed on the pellets to prepare the title pellets.

(2) Preparation of Atorvastatin Prior-Release Tablets

According to the ingredients and contents illustrated in Table 5 below, the title tablets were prepared in the same procedure as in Process (2) of Example 30.

(3) Capsule Filling

The final products of Processes (1) and Process (2) were filled in hydroxypropylmethylcellulose hard capsules using a capsule filling machine (SF-40N, Sejong Pharmatech Co., Ltd., South Korea) to prepare the title capsules each containing both 360 mg of diltiazem and 10.85 mg of atorvastatin.

### Example 36

### Two-Phase Preparation of Diltiazem-Atorvastatin Capsules

(1) Preparation of Diltiazem Delayed-Release Granules

According to the ingredients and contents illustrated in Table 5 below, diltiazem hydrochloride, fumaric acid and hydroxypropylmethylcellulose were sieved through a No. 35 sieve and mixed in a double cone mixer. The mixture was placed in a fluidized bed granulator (GPCG 1, Glatt, Germany). Meanwhile, a poly(ethyl acrylate, methyl methacrylate, trimethylaminoethyl methacrylate) copolymer (Eudragit RS PO, Degussa, Germany) was suspended in purified water (100 mg) to prepare a binding solution. The binding solution was sprayed thereon to form granules, followed by drying. A solution of hydroxypropylmethylcellulose phthalate dissolved in a 1:1 (200 mg:200 mg) mixture of ethanol and methylene chloride was sprayed and coated on the granules to prepare the title granules.

(2) Preparation of Atorvastatin Prior-Release Granules

According to the ingredients and contents illustrated in Table 5 below, the title granules were prepared in the same procedure as in Process (2) of Example 31.

(3) Mixing and Filling

The final products of Processes (1) and Process (2) were mixed in a double cone mixer at room temperature, and croscarmellose sodium given in a prior-release compartment for Example 36 of Table 5 was added thereto, followed by mixing using a double cone mixer. Colloidal silicon dioxide was mixed therewith, and magnesium stearate was added thereto, followed by final mixing. The final mixture was placed in a powder feeder and filled in gelatin hard capsules using a capsule filling machine (SF-40N, Sejong Pharmatech Co., Ltd., South Korea) to prepare the title capsules each containing both 360 mg of diltiazem and 10.85 mg of atorvastatin calcium trihydrate.

### Example 37

### Two-Phase Preparation of Diltiazem-Atorvastatin Capsules

(1) Preparation of Diltiazem Delayed-Release Granules

According to the ingredients and contents illustrated in Table 5 below, diltiazem hydrochloride, fumaric acid and hydroxypropylmethylcellulose were sieved through a No. 35 sieve and mixed in a double cone mixer. The mixture was placed in a fluidized bed granulator (GPCG 1, Glatt, Germany), and a binding solution of Eudragit L100 and ethylcellulose dissolved in a 1:1 (200 mg:200 mg) mixture of ethanol and methylene chloride was sprayed thereon to form granules, followed by drying to prepare the title granules.

(2) Preparation of Atorvastatin Prior-Release Granules

According to the ingredients and contents illustrated in Table 5 below, the title granules were prepared in the same procedure as in Process (2) of Example 31.

(3) Mixing and Filling

According to the ingredients and contents illustrated in Table 5 below, the title capsules were prepared in the same procedure as in Process (3) of Example 36.

### Example 38

### Two-Phase Preparation of Diltiazem-Atorvastatin Capsules

(1) Preparation of Diltiazem Delayed-Release Granules

According to the ingredients and contents illustrated in Table 5 below, diltiazem hydrochloride, fumaric acid and polyethylene oxide were sieved through a No. 35 sieve and mixed in a double cone mixer at room temperature. The mixture was placed in a fluidized bed granulator (GPCG 1, Glatt, Germany). Meanwhile, hydroxypropylmethylcellulose was dissolved in purified water (100 mg) to prepare a binding solution. The binding solution was sprayed thereon to form granules, followed by drying. A solution of hydroxypropylmethylcellulose phthalate dissolved in a 1:1 (200 mg:200 mg) mixture of ethanol and methylene chloride was sprayed and coated on the granules to prepare the title granules.

(2) Preparation of Atorvastatin Prior-Release Tablets

According to the ingredients and contents illustrated in Table 5 below, granules were prepared in the same procedure as in Process (2) of Example 30. The granules were then compressed into tablets containing 10.85 mg of atorvastatin calcium trihydrate per tablet, using a rotary tablet press (MRC-30, Sejong Pharmatech Co., Ltd., South Korea).

(3) Capsule Filling

The final products of Processes (1) and (2) were filled in hydroxypropylmethylcellulose hard capsules using a capsule filling machine (SF-40N, Sejong Pharmatech Co., Ltd., South Korea) to prepare the title capsules each containing both 360 mg of diltiazem and 10.85 mg of atorvastatin calcium trihydrate.

### Example 39

### Two-Phase Preparation of Diltiazem-Atorvastatin Capsules

(1) Preparation of Diltiazem Delayed-Release Granules

According to the ingredients and contents illustrated in Table 5 below, diltiazem hydrochloride, fumaric acid and a carboxyvinyl polymer (Carbomer 71G, Lubrizol, USA) were sieved through a No. 35 sieve and mixed in a double cone mixer. The mixture was placed in a high-speed mixer (Lab. Pharma Mixer P, Diosna, Germany) and polyvinyl acetate (Kollicoat SR30D, D-BASF, Germany) were added thereto, followed by kneading. The kneaded material was granulated using an oscillator with a No. 20 sieve, and the granules were dried in a hot-water dryer at 60° C. The granules were sieved again through a No. 20 sieve (KYK-60, KoreaMedi Co., Ltd., South Korea). Magnesium stearate was added thereto, followed by final mixing in a double cone mixer to prepare the title granules.

(2) Preparation of Atorvastatin Prior-Release Tablets

According to the ingredients and contents illustrated in Table 5 below, the title tablets were prepared in the same procedure as in Process (2) of Example 38.

(3) Capsule Filling

Using the final products of Processes (1) and (2), the title capsules were prepared in the same procedure as in Process (3) of Example 38.

### Example 40

### Preparation of Diltiazem-Atorvastatin Multi-Layered Tablets

(1) Preparation of Diltiazem Delayed-Release Layer

According to the ingredients and contents illustrated in Table 6 below, diltiazem hydrochloride, fumaric acid and a carboxyvinyl polymer (Carbomer 71G, Lubrizol, USA) were sieved through a No. 35 sieve and mixed in a double cone mixer. The mixture was placed in a high-speed mixer (Lab. Pharma Mixer P, Diosna, Germany) and polyvinyl acetate (Kollicoat SR30D, D-BASF, Germany) was added thereto, followed by mixing. The mixture was granulated using an oscillator with No. 20 sieve, and dried in a hot-water dryer at 60° C. The granules were sieved again through a No. 20 sieve (KYK-60, KoreaMedi Co., Ltd., South Korea). Magnesium stearate was added thereto, followed by final mixing in a double cone mixer to prepare the title layer.

(2) Preparation of Atorvastatin Prior-Release Layer

According to the ingredients and contents illustrated in Table 6 below, the title layer was prepared in the same manner as in Process (2) of Example 30 (atorvastatin strontium anhydride was used instead of atorvastatin calcium trihydrate in Process (2) of Example 30).

(3) Compression and Coating

Using the final products of Processes (1) and (2), the title multi-layered tablets were prepared in the same ° C. as in Process (3) of Example 30.

### Example 41

### Preparation of Diltiazem-Atorvastatin Multi-Layered Tablets

(1) Preparation of Diltiazem Delayed-Release Layer

According to the ingredients and contents illustrated in Table 6 below, diltiazem hydrochloride, microcrystalline cellulose, Kollidon 30, and fumaric acid were sieved through a No. 35 sieve and mixed in a double cone mixer. The mixture and purified water (30 mg) were placed and kneaded in a high-speed mixer (Lab. Pharma Mixer P, Diosna, Germany). The kneaded material was extruded through an extruder (EXDCS-100, Fuji Denki Kogyo Company, Japan) and the extrudate was Spheronized into an appropriate size. The spheroidized material was dried in a hot-water dryer at 60° C. and sieved through a No. 25 sieve. Meanwhile, hydroxypropylmethylcellulose2910, titanium oxide, talc, polysorbate 80, and a simethicone emulsion were mixed, and Eudragit NE 30D was added thereto to prepare a coating solution. After the preparation of the coating solution was complete, the beads were placed in a fluidized bed coater (GPCG-1, Glatt, Germany) and were coated with the coating solution to an appropriate thickness (about 0.05 mm) The coated beads were dried in an oven at 45° C. Eudragit L100, Kollidon SR, and magnesium stearate were added thereto, followed by final mixing in a double cone mixer (Dasan Pharmatech, South Korea), thereby preparing the title layer.

(2) Preparation of Atorvastatin Prior-Release Layer

According to the ingredients and contents illustrated in Table 6 below, the title layer was prepared in the same procedure as in Process (2) of Example 30 (atorvastatin calcium anhydride was used instead of atorvastatin calcium trihydrate in Process (2) of Example 30).

(3) Compression and Coating

Using the final products of Processes (1) and (2), the title multi-layered tablets were prepared in the same procedure as in Process (3) of Example 30.

### Example 42

### Preparation of Diltiazem-Atorvastatin Two-Phase Matrix Tablet

(1) Preparation of Diltiazem Delayed-Release Granules

According to the ingredients and contents illustrated in Table 6 below, diltiazem hydrochloride, fumaric acid and hydroxypropylmethylcellulose were sieved through a No. 35 sieve and mixed. The mixture was placed in a high-speed mixer (Lab. Pharma Mixer P, Diosna, Germany) and Kollicoat SR30D was added thereto, followed by kneading. Then, kneaded material was sieved using an oscillator with a No. 20 sieve, and the granules were dried in a hot-water dryer at 60° C. After completion of the drying process, the granules were sieved again through a No. 20 sieve (KYK-60, KoreaMedi Co., Ltd., South Korea) to prepare the title granules. A solution of hydroxypropylmethylcellulose phthalate dissolved in a 1:1 (200 mg:200 mg) mixture of ethanol and methylene chloride was sprayed and coated on the granules to prepare the title granules.

(2) Preparation of Atorvastatin Prior-Release Granules

According to the ingredients and contents illustrated in Table 6 below, the title granules were prepared in the same procedure as in Process (2) of Example 31 (atorvastatin calcium anhydride was used instead of atorvastatin calcium trihydrate in Process (2) of Example 31).

(3) Post-Mixing, Compression and Coating

The final products of Processes (1) and (2) were mixed in a double cone mixer, and then mixed with croscarmellose sodium and colloidal silicon dioxide. Magnesium stearate was added thereto, followed by final mixing in the double cone mixer.

The final mixture was compressed into tablets using a rotary tablet press. Using a Hi-coater (SFC-30N, Sejong Pharmatech Co., Ltd., South Korea), a film-coating layer was formed on the compressed tablets to prepare the title two-phase matrix tablets.

### Example 43

### Preparation of Diltiazem-Atorvastatin Two-Phase Matrix Tablets

(1) Preparation of Diltiazem Delayed-Release Granules

According to the ingredients and contents illustrated in Table 6 below, diltiazem hydrochloride, fumaric acid and hydroxypropylmethylcellulose were sieved through a No. 35 sieve and mixed in a double cone mixer. The mixture was placed in a fluidized bed granulator (GPCG 1, Glatt, Germany). Meanwhile, ethylcellulose was dissolved in ethanol (50 mg) to prepare a binding solution which was then sprayed thereon to form granules, followed by drying. A solution of Eudragit RS PO dissolved in a 1:1 (200 mg:200 mg) mixture of ethanol and methylene chloride was sprayed and coated on the granules to prepare the title granules.

(2) Preparation of Atorvastatin Prior-Release Granules

According to the ingredients and contents illustrated in Table 6 below, the title granules were prepared in the same procedure as in Process (2) of Example 31 (atorvastatin calcium anhydride was used instead of atorvastatin calcium trihydrate in Process (2) of Example 31).

(3) Post-Mixing, Compression and Coating

Using the final products of Processes (1) and (2), the title tablets were prepared in the same procedure as in Process (3) of Example 42.

### Example 44

### Preparation of Diltiazem-Atorvastatin Multi-Layered Tablets

(1) Preparation of Diltiazem Delayed-Release Layer

According to the ingredients and contents illustrated in Table 6 below, diltiazem hydrochloride, fumaric acid and hydroxypropylmethylcellulose were sieved through a No. 35 sieve and mixed in a double cone mixer. The mixture was placed in a fluidized bed granulator (GPCG 1, Glatt, Germany). Meanwhile, ethylcellulose was dissolved in ethanol (50 mg) to prepare a binding solution which was then sprayed thereon to form granules, followed by drying. A solution of hydroxypropylmethylcellulose phthalate dissolved in a 1:1 (200 mg:200 mg) mixture of ethanol and methylene chloride was sprayed and coated on the granules. Magnesium stearate was added thereto, followed by final mixing in a double cone mixer to prepare the title layer.

(2) Preparation of Atorvastatin Prior-Release Layer

According to the ingredients and contents illustrated in Table 6 below, the title layer was prepared in the same manner as in Process (2) of Example 40.

(3) Compression and Coating

Using the final products of Processes (1) and (2), the title multi-layered tablet preparation was prepared in the same procedure as in Process (3) of Example 30.

### Example 45

### Two-Phase Preparation of Diltiazem-Atorvastatin Capsules

(1) Preparation of Diltiazem Delayed-Release Pellets

According to the ingredients and contents illustrated in Table 6 below, diltiazem hydrochloride, fumaric acid, and hydroxypropylmethylcellulose were sieved through a No. 35 sieve and were placed together with sugar seeds (sugar spheres) in a fluidized bed granulator (GPCG 1, Glatt, Germany), followed by mixing. Meanwhile, hydroxypropylmethylcellulose was dissolved in purified water (100 mg) to prepare a binding solution. The binding solution was sprayed thereon to form diltiazem-containing pellets. A polyvinyl acetate 30% suspension (Kollicoat SR 30D, D-BASF, Germany) was sprayed on the pellets which were then dried. A solution of hydroxypropylmethylcellulose phthalate dissolved in a 1:1 (200 mg:200 mg) mixture of ethanol and methylene chloride was sprayed on the pellets to prepare the title pellets.

(2) Preparation of Atorvastatin Prior-Release Granules

According to the ingredients and contents illustrated in Table 6 below, the title granules were prepared in the same procedure as in Process (2) of Example 30 (atorvastatin strontium anhydride was used instead of atorvastatin calcium trihydrate in Process (2) of Example 30).

(3) Mixing and Capsule Filling

Using the final products of Processes (1) and (2), the title capsules were prepared in the same procedure as in Process (3) of Example 36.

### Example 46

### Two-Phase Preparation of Diltiazem-Atorvastatin Capsules

(1) Preparation of Diltiazem Delayed-Release Pellets

According to the ingredients and contents illustrated in Table 6 below, diltiazem hydrochloride, fumaric acid, hydroxypropylmethylcellulose were sieved through a No. 35 sieve and were placed together with sugar seeds (sugar spheres) in a fluidized bed granulator (GPCG 1, Glatt, Germany), followed by mixing. Meanwhile, hydroxypropylmethylcellulose was dissolved in purified water (100 mg) to prepare a binding solution. The binding solution was sprayed thereon to form diltiazem-containing pellets. Polyvinyl acetate (Kollicoat SR30D, D-BASF, Germany) was sprayed on the diltiazem-containing pellets which were then dried. A solution of a polymethacrylic acid copolymer (Eudragit L100, Degussa, Germany) dissolved in a 1:1 (200 mg:200 mg) mixture of ethanol and methylene chloride was sprayed on the pellets to prepare the title pellets.

(2) Preparation of Atorvastatin Prior-Release Tablets

According to the ingredients and contents illustrated in Table 6 below, the title tablets were prepared in the same procedure as in Process (2) of Example 38.

(3) Capsule Filling

The final products of Processes (1) and (2) were filled in hydroxypropylmethylcellulose hard capsules using a capsule filling machine (SF-40N, Sejong Pharmatech Co., Ltd., South Korea) to prepare the title capsules each containing both 360 mg of diltiazem and 11.595 mg of atorvastatin strontium anhydride.

TABLE 5

| | | Composition ratio (mg/tablet) | | | | | | | | | |
|---|---|---|---|---|---|---|---|---|---|---|---|
| | | Example No. | | | | | | | | | |
| Ingredients | | 30 | 31 | 32 | 33 | 34 | 35 | 36 | 37 | 38 | 39 |
| Delayed-release | Diltiazem | 360 | 360 | 360 | 360 | 360 | 360 | 360 | 360 | 360 | 360 |
| compartment | hydrochloride | | | | | | | | | | |
| | Microcrystalline | 44.4 | | | | | | | | | |
| | cellulose | | | | | | | | | | |
| | Fumaric acid | 90 | 90 | 90 | 90 | 75 | 75 | 90 | 90 | 90 | 90 |
| | Kollidon 30¹⁾ | 6 | | | | | | | | | |
| | Polyethylene | | | | | | | | | 71 | |
| | oxide | | | | | | | | | | |
| | Sugar seed | | | | | 72.5 | 72.5 | | | | |
| | Hydroxypropyl | 2.5 | 71 | 71 | 71 | 42.5 | 42.5 | 60 | 60 | 20 | |
| | methylcellulose | | | | | | | | | | |
| | Kollicoat SR30D²⁾ | | 110 | | | 90 | 90 | | | | 90 |
| | Eudragit RS PO³⁾ | | | 90 | | | | 90 | | | |
| | Eudragit L100⁴⁾ | 50 | | | | | 90 | | 100 | | |
| | Eudragit NE 30D⁵⁾ | 52 | | | | | | | | | |
| | Carbomer 71G⁶⁾ | | | | | | | | | | 91 |
| | Kollidon SR⁷⁾ | 50 | | | | | | | | | |
| | Ethylcellulose | | | 20 | 20 | | | | 50 | | |
| | Hydroxypropyl | | 30 | | 90 | 90 | | 30 | | 90 | |
| | methylcellulose | | | | | | | | | | |
| | phthalate | | | | | | | | | | |
| | Talc | 10 | | | | | | | | | |
| | Titanium oxide | 2 | | | | | | | | | |
| | Polysorbate 80 | 0.05 | | | | | | | | | |
| | Simethicone | 0.05 | | | | | | | | | |
| | emulsion | | | | | | | | | | |
| | Ethanol (volatile) | | 200 | 250 | 250 | 200 | 200 | 200 | 200 | 200 | |
| | Methylene chloride | | 200 | 200 | 200 | 200 | 200 | 200 | 200 | 200 | |
| | (volatile) | | | | | | | | | | |
| | Purified water | 30 | | | | 100 | 100 | 100 | | 100 | |
| | (volatile) | | | | | | | | | | |
| | Magnesium stearate | 5 | | | 5 | | | | | | 5 |
| Prior-release | Atorvastatin calcium | 10.85 | 10.85 | 10.85 | 10.85 | 10.85 | 10.85 | 10.85 | 10.85 | 10.85 | 10.85 |
| compartment | trihydrate | | | | | | | | | | |
| | Microcrystalline | 190.25 | 107.15 | 107.15 | 105.65 | 48.65 | 48.65 | 88.65 | 58.65 | 87.65 | 82.65 |
| | cellulose | | | | | | | | | | |
| | Precipitated calcium | 80 | 80 | 80 | 80 | 80 | 80 | 80 | 80 | 80 | 80 |
| | carbonate | | | | | | | | | | |
| | Lactose | 98 | 98 | 98 | 98 | 50 | 50 | 50 | 50 | 50 | 50 |
| | Croscarmellose | 40 | 40 | 40 | 40 | 40 | 40 | 40 | 40 | 40 | 40 |
| | sodium | | | | | | | | | | |
| | Hydroxypropylcellulose | 5 | 5 | 5 | 5 | 5 | 5 | 5 | 5 | 5 | 5 |
| | Sodium lauryl | 10 | 10 | 10 | 10 | 10 | 10 | 10 | 10 | 10 | 10 |
| | sulfate | | | | | | | | | | |
| | Pregelatinized | 43 | 43 | 43 | 43 | 43 | 43 | 43 | 43 | 43 | 43 |
| | starch | | | | | | | | | | |
| | Colloidal silicon | 1 | 1 | 1 | 1 | 1 | 1 | 1 | 1 | 1 | 1 |
| | dioxide⁸⁾ | | | | | | | | | | |
| | Magnesium stearate | 2.9 | 5 | 5 | 1.5 | 1.5 | 1.5 | 1.5 | 1.5 | 1.5 | 1.5 |
| Coating | Hydroxypropyl | 2.6 | 2.6 | 2.6 | 2.6 | | | | | | |
| layer | methylcellulose | | | | | | | | | | |
| | 2910 | | | | | | | | | | |
| | Hydroxypropyl | 2.6 | 2.6 | 2.6 | 2.6 | | | | | | |
| | cellulose | | | | | | | | | | |
| | Titanium oxide | 2.3 | 2.3 | 2.3 | 2.3 | | | | | | |
| | Talc | 1.5 | 1.5 | 1.5 | 1.5 | | | | | | |
| | Ethanol (volatile) | 255 | 255 | 255 | 255 | | | | | | |
| | Purified water | 63.75 | 63.75 | 63.75 | 63.75 | | | | | | |
| | (volatile) | | | | | | | | | | |

| Total | 1080 | 1070 | 1040 | 1070 | 1010 | 1010 | 950 | 950 | 950 | 950 |
|---|---|---|---|---|---|---|---|---|---|---|
| Oral formulation | Multi- | Tablet | Tablet | Multi- | Capsule | Capsule | Capsule | Capsule | Capsule | Capsule |
| | layered | | | layered | | | | | | |
| | tablet | | | tablet | | | | | | |

¹⁾Kollidon 30 - Main ingredient: Povidone K30 (BASF)
²⁾Kollicoat SR30D - Main ingredient: polyvinyl acetate 30% suspension (BASF)
³⁾Eudragit RS PO - Main ingredient: poly(ethyl acrylate, methyl methacrylate, trimethylaminoethyl methacrylate) copolymer (Degussa)
⁴⁾Eudragit L100 - Main ingredient: polymethacrylate copolymer (Degussa)
⁵⁾Eudragit NE 30D - Main ingredient: polymethyl methacrylate ethyl acrylate copolymer (Degussa)
⁶⁾Carbomer 71G - Main ingredient: Carboxyvinyl polymer (Lubrizol)
⁷⁾Kollidon SR - Main ingredient: polyvinyl acetate/povidone (BASF)
⁸⁾Colloidal silicon dioxide - trade name: Aerosil 200 (Degussa)

TABLE 6

| | | Composition ratio (mg/tablet) | | | | | | |
|---|---|---|---|---|---|---|---|---|
| | | Example No. | | | | | | |
| Ingredients | | 40 | 41 | 42 | 43 | 44 | 45 | 46 |
| Delayed-release | Diltiazem | 360 | 360 | 360 | 360 | 360 | 360 | 360 |
| compartment | hydrochloride | | | | | | | |
| | Microcrystalline | | 44.4 | | | | | |
| | cellulose | | | | | | | |
| | Fumaric acid | 90 | 90 | 90 | 90 | 90 | 75 | 75 |
| | Kollidon 30¹⁾ | | 6 | | | | | |
| | Sugar seed | | | | | | 72.5 | 72.5 |
| | Hydroxypropyl | | 2.5 | 71 | 71 | 71 | 42.5 | 42.5 |
| | Methyl cellulose | | | | | | | |
| | Kollicoat SR30D²⁾ | 90 | | 110 | | | 90 | 90 |
| | Eudragit RS PO³⁾ | | | | 90 | | | |
| | Eudragit L100⁴⁾ | | 50 | | | | | 90 |
| | Eudragit NE 30D⁵⁾ | | 52 | | | | | |
| | Carbomer 71G⁶⁾ | 91 | | | | | | |
| | Kollidon SR⁷⁾ | | 50 | | | | | |
| | Ethylcellulose | | | | 20 | 50 | | |
| | Hydroxypropylmethyl- | | | 30 | | 90 | 90 | |
| | cellulose phthalate | | | | | | | |
| | Talc | | 10 | | | | | |
| | Titanium oxide | | 2 | | | | | |
| | Polysorbate 80 | | 0.05 | | | | | |
| | Simethicone emulsion | | 0.05 | | | | | |
| | Ethanol (volatile) | | | 200 | 250 | 250 | 200 | 200 |
| | Methylene chloride | | | 200 | 200 | 200 | 200 | 200 |
| | (volatile) | | | | | | | |
| | Purified water | | 30 | | | | 100 | 100 |
| | (volatile) | | | | | | | |
| | Magnesium stearate | 5 | 5 | | | 5 | | |
| Prior-release | Atorvastatin calcium | | 10.35 | 10.35 | 10.35 | | | |
| compartment | anhydride | | | | | | | |
| | Atorvastatin strontium | 11.595 | | | | 11.595 | 11.595 | 11.595 |
| | anhydride | | | | | | | |
| | Microcrystalline | 104.905 | 108.75 | 107.65 | 107.65 | 104.905 | 47.905 | 47.905 |
| | cellulose | | | | | | | |
| | Precipitated calcium | 80 | 80 | 80 | 80 | 80 | 80 | 80 |
| | carbonate | | | | | | | |
| | Lactose | 98 | 98 | 98 | 98 | 98 | 50 | 50 |
| | Croscarmellose sodium | 40 | 40 | 40 | 40 | 40 | 40 | 40 |
| | Hydroxypropylcellulose | 5 | 5 | 5 | 5 | 5 | 5 | 5 |
| | Sodium lauryl sulfate | 10 | 10 | 10 | 10 | 10 | 10 | 10 |
| | Pregelatinized starch | 43 | 43 | 43 | 43 | 43 | 33 | 33 |
| | Colloidal silicon | 1 | 1 | 1 | 1 | 1 | 1 | 1 |
| | dioxide⁸⁾ | | | | | | | |
| | Magnesium stearate | 1.5 | 2.9 | 5 | 5 | 1.5 | 1.5 | 1.5 |
| Coating | Hydroxypropylmethyl- | 2.6 | 2.6 | 2.6 | 2.6 | 2.6 | | |
| layer | cellulose 2910 | | | | | | | |
| | Hydroxypropylcellulose | 2.6 | 2.6 | 2.6 | 2.6 | 2.6 | | |
| | Titanium oxide | 2.3 | 2.3 | 2.3 | 2.3 | 2.3 | | |
| | Talc | 1.5 | 1.5 | 1.5 | 1.5 | 1.5 | | |
| | Ethanol (volatile) | 255 | 255 | 255 | 255 | 255 | | |
| | Purified water | 63.75 | 63.75 | 63.75 | 63.75 | 63.75 | | |
| | (volatile) | | | | | | | |

| Total | 1040 | 1080 | 1040 | 1040 | 1070 | 1010 | 1010 |
|---|---|---|---|---|---|---|---|
| Oral formulation | Multi- | Multi- | Tablet | Tablet | Multi- | Capsule | Capsule |
| | layered | layered | | | layered | | |
| | tablet | tablet | | | tablet | | |

¹⁾Kollidon 30 - Main ingredient: Povidone K30 (BASF)
²⁾Kollicoat SR30D - Main ingredient: polyvinyl acetate 30% suspension (BASF)
³⁾Eudragit RS PO - Main ingredient: poly(ethyl acrylate, methyl methacrylate, trimethylaminoethyl methacrylate) copolymer (Degussa)
⁴⁾Eudragit L100 - Main ingredient: polymethacrylate copolymer (Degussa)
⁵⁾Eudragit NE 30D - Main ingredient: polymethyl methacrylate ethyl acrylate copolymer (Degussa)
⁶⁾Carbomer 71G - Main ingredient: Carboxyvinyl polymer (Lubrizol)
⁷⁾Kollidon SR - Main ingredient: polyvinyl acetate/povidone (BASF)
⁸⁾Colloidal silicon dioxide - trade name: Aerosil 200 (Degussa)

### Experimental Example 1

### Comparative Dissolution Profile Test for Diltiazem/Simvastatin Tablets of the Present Invention

A comparative dissolution profile test was performed using diltiazem hydrochloride/simvastatin tablets prepared in Example 1 and control drugs (Zocor: simvastatin single drug, MSD, Cardizem LA: diltiazem single drug, Biovail Corporation). In the case of dissolution profile test of a diltiazem ingredient, the dissolution medium was changed from a simulated gastric juice to a simulated intestinal juice 2 hours after the start of the test. Details of the dissolution profile test of each ingredient were as follows. The results obtained are illustrated in FIG. 1.

Test Method for diltiazem:

Based on the general dissolution test method described in the Korean Pharmacopoeia (8^{th} revision)

Test method: Paddle method, 75 rpm (USP 30 Diltiazem HCl extended release tablet)

Dissolution medium: 0.01 M hydrochloric acid solution 750 mL (0 to 2 hours), pH 6.8 simulated intestinal juice 1,000 mL (after 2 hours)

Analysis method: UV-Vis spectrophotometry (detection wavelength=240 nm).

Test Method for Simvastatin:

Based on the “Simvastatin tablet” part in the United States Pharmacopoeia (USP 29)

Test method: Paddle method, 50 rpm

Dissolution medium: pH 7.0 buffer solution (composition=0.01 M sodium dihydrogen phosphate solution containing sodium lauryl sulfate 0.5% wt/wt as surfactant), 900 mL

Analysis method: UV-Vis spectrophotometry (detection wavelength=maximum 247 nm and minimum 257 nm).

As seen in FIG. 1, the simvastatin ingredient of the prior-release compartment of the present invention showed a dissolution profile substantially equal to that of the control drug Zocor, but the diltiazem ingredient of the delayed-release compartment showed delayed initiation of dissolution as compared to that of the control drug Cardizem LA. In the case of the diltiazem hydrochloride/simvastatin multi-layered tablet according to the present invention, dissolution rates of the diltiazem ingredient were all within 10% until 3 hours after the test began, which were far lower than those of the control drugs (about 20%).

As described above, the diltiazem hydrochloride/simvastatin multi-layered tablets according to the present invention exhibit delayed initiation of diltiazem release after the completion of simvastatin release unlike the control drugs (i.e., diltiazem single drugs), and therefore diltiazem may be less likely to be metabolized in the liver ahead of simvastatin.

### Experimental Example 2

### Comparative Dissolution Profile Test for Diltiazem/Simvastatin Capsules of the Present Invention

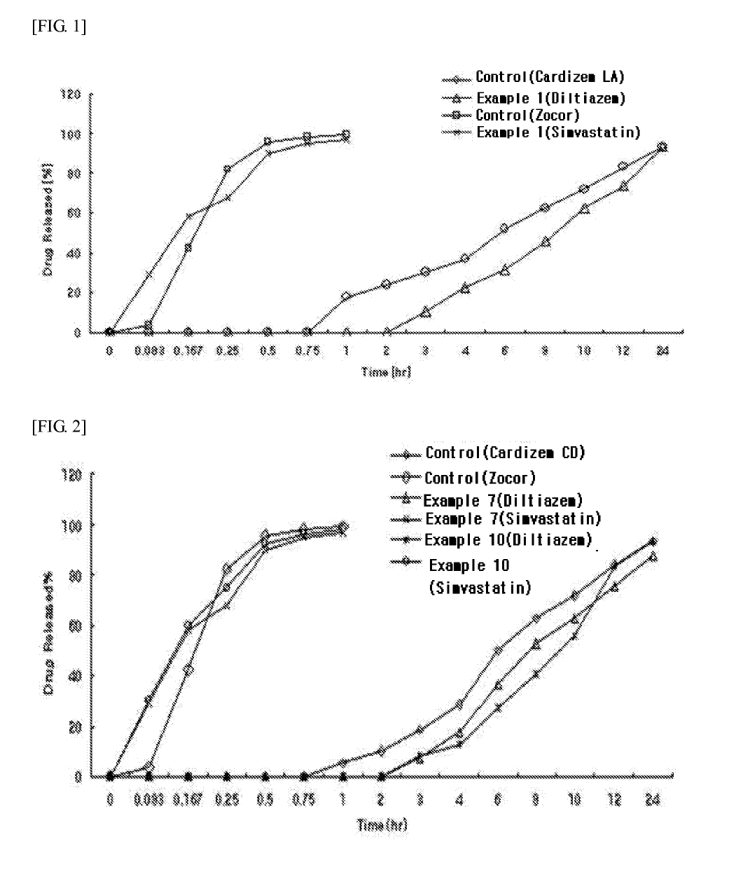

A comparative dissolution profile test was performed using diltiazem hydrochloride/simvastatin capsules prepared in Examples 7 and 10 and control drugs (Zocor: simvastatin single drug, MSD, Cardizem CD: diltiazem single drug, Biovail Corporation). In the case of dissolution profile test of a diltiazem ingredient, the dissolution medium was changed from a simulated gastric juice to a simulated intestinal juice 2 hours after the start of the test. Details of the dissolution profile test of each ingredient were as follows. The results obtained are illustrated in FIG. 2.

Test Method for Diltiazem:

Based on the general dissolution test method described in the Korean Pharmacopoeia (8^{th} revision)

Test method: Paddle method, 75 rpm (USP 30 Diltiazem HCl extended release tablet)

Dissolution medium: 0.01 M hydrochloric acid solution 750 mL (0 to 2 hours), pH 6.8 simulated intestinal juice 1,000 mL (after 2 hours)

Analysis method: UV-Vis spectrophotometry (detection wavelength=240 nm).

Test Method for Simvastatin:

Based on the “Simvastatin tablet” part in USP 29

Test method: Paddle method, 50 rpm

Dissolution medium: pH 7.0 buffer solution (composition=0.01 M sodium dihydrogen phosphate solution containing sodium lauryl sulfate 0.5% wt/wt as surfactant), 900 mL

Analysis method: UV-Vis spectrophotometry (detection wavelength=maximum 247 nm and minimum 257 nm).

As seen in FIG. 2, the simvastatin ingredient of the prior-release compartment of the present invention showed a dissolution profile substantially equal to that of the control drug Zocor, but the diltiazem ingredient of the delayed-release compartment showed delayed initiation of dissolution as compared to that of the control drug Cardizem CD. In the case of the diltiazem hydrochloride/simvastatin capsules according to the present invention, dissolution rates of the diltiazem ingredient were all within 10% until 3 hours after the test began, which were far lower than those of the control drugs (about 20%).

As described above, the diltiazem hydrochloride/simvastatin capsules according to the present invention exhibit delayed initiation of diltiazem release after the completion of simvastatin release unlike the control drugs (i.e., diltiazem single drugs), and therefore diltiazem may be less likely to be metabolized in the liver ahead of simvastatin.

### Experimental Example 3

### Comparative Dissolution Profile Test for Diltiazem/Lovastatin Tablets of the Present Invention

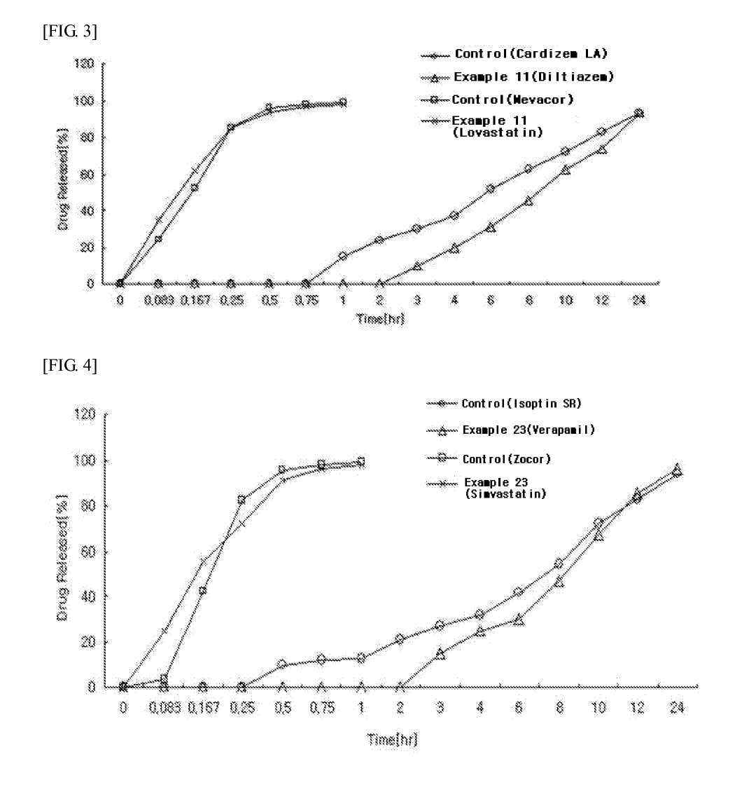

A comparative dissolution profile test was performed using diltiazem hydrochloride/lovastatin tablets prepared in Example 11 and control drugs (Mevacor: lovastatin single drug, MSD, Cardizem LA: diltiazem single drug, Biovail Corporation). In the case of dissolution profile test of a diltiazem ingredient, the dissolution medium was changed from a simulated gastric juice to a simulated intestinal juice 2 hours after the start of the test. Details of the dissolution profile test of each ingredient were as follows. The results obtained are illustrated in FIG. 3.

Test Method for Diltiazem:

Based on the general dissolution test method described in the Korean Pharmacopoeia (8^{th} revision)

Test method: Paddle method, 75 rpm (USP 30 Diltiazem HCl extended release tablet)

Dissolution medium: 0.01 M hydrochloric acid solution 750 mL (0 to 2 hours), pH 6.8 simulated intestinal juice 1,000 mL (after 2 hours)

Analysis method: UV-Vis spectrophotometry (detection wavelength=240 nm).

Test Method for Lovastatin:

Based on the “Lovastatin tablet” part in USP 29

Test method: Paddle method, 50 rpm

Dissolution medium: pH 7.0 buffer solution (composition=0.01 M sodium dihydrogen phosphate solution containing sodium lauryl sulfate 2% wt/wt as surfactant), 900 mL

Analysis method: High performance liquid chromatography

Detection wavelength: 230 nm

Mobile phase: acetonitrile: 0.02 M sodium dihydrogen phosphate buffer (pH=4.0): methanol=5:3:1.

Column: stainless column (having an inner diameter of 4 6 mm and a length of 250 mm) packed with octadecyl silyl silica gel.

Flow rate: 1.5 mL/min.

As seen in FIG. 3, the lovastatin ingredient of the prior-release compartment of the present invention showed a dissolution profile substantially equal to that of the control drug Mevacor, but the diltiazem ingredient of the delayed-release compartment showed delayed initiation of dissolution as compared to that of the control drug Cardizem LA. In the case of the diltiazem hydrochloride/lovastatin tablet according to the present invention, dissolution rates of the diltiazem ingredient were all within 10% until 3 hours after the test began, which were far lower than those of the control drugs (about 20%).

As described above, the diltiazem hydrochloride/lovastatin tablets according to the present invention exhibit delayed initiation of diltiazem release after the completion of lovastatin release unlike the control drugs (i.e., diltiazem single drugs), and therefore diltiazem may be less likely to be metabolized in the liver ahead of lovastatin.

### Experimental Example 4

### Comparative Dissolution Profile Test for Verapamil/Simvastatin Tablets of the Present Invention

A comparative dissolution profile test was performed using verapamil hydrochloride/simvastatin tablets prepared in Example 23 and control drugs (Zocor: simvastatin single drug, MSD, Isoptin SR: verapamil single drug, Ranbaxy Laboratories Ltd.). In the case of dissolution profile test of a verapamil ingredient, the dissolution medium was changed from a simulated gastric juice to a simulated intestinal juice 2 hours after the start of the test. Details of the dissolution profile test of each ingredient were as follows. The results obtained are illustrated in FIG. 4.

Test Method for Verapamil:

Based on the general dissolution test method described in the Korean Pharmacopoeia (8^{th} revision)

Test method: Paddle method, 50 rpm

Dissolution medium: 0.01 M hydrochloric acid solution 750 mL (0 to 2 hours), pH 6.8 simulated intestinal juice 1,000 mL (after 2 hours)

Analysis method: UV-Vis spectrophotometry (detection wavelength=maximum 278 nm and minimum 300 nm).

Test Method for Simvastatin:

Based on the “Simvastatin tablet” part in USP 30

Test method: Paddle method, 50 rpm

Dissolution medium: pH 7.0 buffer solution (composition=0.01 M sodium dihydrogen phosphate solution containing sodium lauryl sulfate 0.5% wt/wt as surfactant), 900 mL

Analysis method: UV-Vis spectrophotometry (detection wavelength=maximum 247 nm and minimum 257 nm).

As seen in FIG. 4, the simvastatin ingredient of the prior-release compartment of the present invention showed a dissolution profile substantially equal to that of the control drug Zocor, but the verapamil ingredient of the delayed-release compartment showed delayed initiation of dissolution as compared to that of the control drug Isoptin SR. In the case of the verapamil hydrochloride/simvastatin tablet according to the present invention, dissolution rates of the verapamil ingredient were all within 20% until 3 hours after the test began, which were far lower than those of the control drugs (about 30%).

As described above, the verapamil hydrochloride/simvastatin tablets according to the present invention exhibit delayed initiation of verapamil release after the completion of simvastatin release unlike the control drugs (i.e., verapamil single drugs), and therefore verapamil may be less likely to be metabolized in the liver ahead of simvastatin.

### Experimental Example 5

### Comparative Dissolution Profile Test for Verapamil/Pravastatin Capsules of the Present Invention

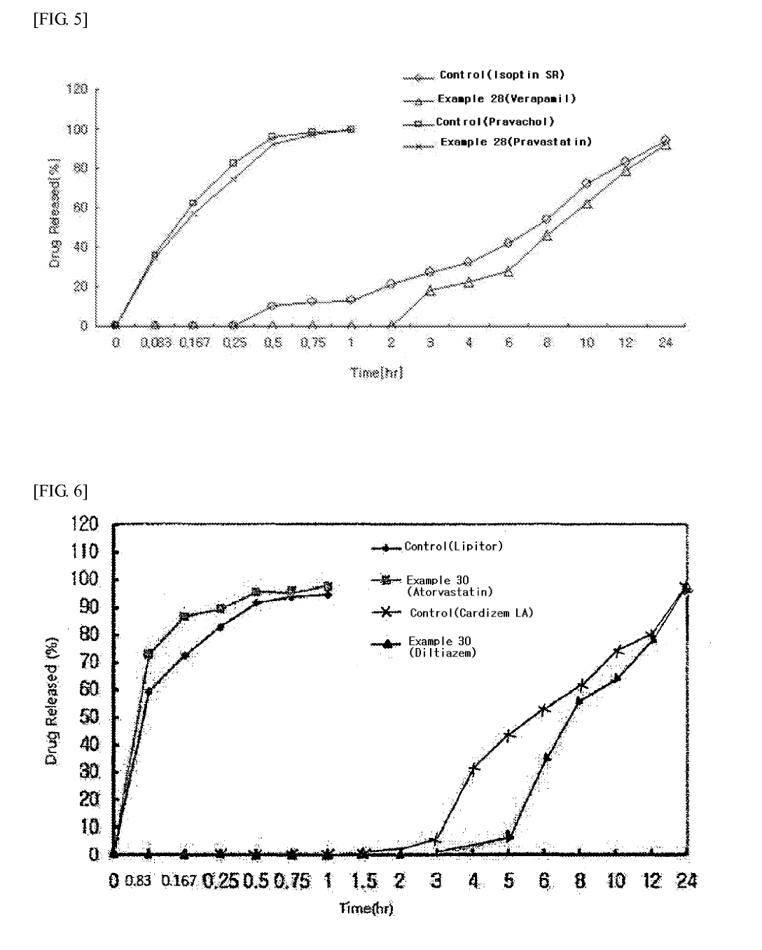

A comparative dissolution profile test was performed using verapamil hydrochloride/pravastatin capsules prepared in Example 28 and control drugs (Pravachol: pravastatin single drug, MSD, Isoptin SR: verapamil single drug, Ranbaxy Laboratories Ltd.). In the case of dissolution profile test of a verapamil ingredient, the dissolution medium was changed from a simulated gastric juice to a simulated intestinal juice 2 hours after the start of the test. Details of the dissolution profile test of each ingredient were as follows. The results obtained are illustrated in FIG. 5.

Test Method for Verapamil:

Based on the general dissolution test method described in the Korean Pharmacopoeia (8^{th} revision)

Test method: Paddle method, 50 rpm

Dissolution medium: 0.01 M hydrochloric acid solution 750 mL (0 to 2 hours), pH 6.8 simulated intestinal juice 1,000 mL (after 2 hours)

Analysis method: UV-Vis spectrophotometry (detection wavelength=maximum 278 nm and minimum 300 nm).

Test Method for Pravastatin:

Based on the general dissolution test method described in the Korean Pharmacopoeia (8^{th} revision)

Test method: Paddle method, 50 rpm

Dissolution medium: purified water, 900 mL

Analysis method: High performance liquid chromatography

Detection wavelength: 239 nm

Mobile phase: acetonitrile:0.05 M phosphate buffer (pH=2.3)=56:44

Column: stainless column (having an inner diameter of 4.6 mm and a length of 250 mm) packed with octadecyl silyl silica gel.

Flow rate: 1.5 mL/min.

As seen in FIG. 5, the pravastatin ingredient of the prior-release compartment of the present invention showed a dissolution profile substantially equal to that of the control drug Pravachol, but the verapamil ingredient of the delayed-release compartment showed delayed initiation of dissolution as compared to that of the control drug Isoptin SR. In the case of the verapamil hydrochloride/pravastatin capsules according to the present invention, dissolution rates of the verapamil ingredient were all within 20% until 3 hours after the test began, which were far lower than those of the control drugs (about 30%).

As described above, the verapamil hydrochloride/pravastatin capsules according to the present invention exhibit delayed initiation of verapamil release after the completion of pravastatin release unlike the control drugs (i.e., verapamil single drugs), and therefore verapamil may be less likely to be metabolized in the liver ahead of pravastatin.

### Experimental Example 6

### Comparative Dissolution Profile Test for Diltiazem/Atorvastatin Pharmaceutical Preparation of the Present Invention

A comparative dissolution profile test was performed using diltiazem hydrochloride/atorvastatin multi-layered tablets prepared in Example 30 of the present invention and control drugs (Lipitor: atorvastatin single drug, Pfeizer Inc., Cardizem LA: diltiazem single drug, Biovail Corporation), diltiazem hydrochloride/atorvastatin capsules prepared in Examples 37 and 45, and control drugs (Lipitor: atorvastatin single drug, Pfeizer Inc., Cardizem CD: diltiazem single drug, Biovail Corporation). In the case of dissolution profile test of a diltiazem ingredient, the dissolution medium was changed from a simulated gastric juice (0.01 M hydrochloric acid solution) to a simulated intestinal juice (pH 6.8) 2 hours after the start of the test. Details of the dissolution profile test of each ingredient were as follows.

Test Method for Diltiazem:

Based on the general dissolution test method described in the Korean Pharmacopoeia (8^{th} revision)

Test method: Paddle method, 75 rpm (USP 30 Diltiazem HCl extended release tablet)

Dissolution medium: 0.01 M hydrochloric acid solution 750 mL (0 to 2 hours), pH 6.8 simulated intestinal juice 1,000 mL (after 2 hours)

Analysis method: UV analyzer

Test Method for Atorvastatin:

Based on the general dissolution test method described in the Korean Pharmacopoeia (8^{th} revision)

Test method: Paddle method, 50 rpm

Dissolution medium: pH 7.0 buffer solution (composition=0.01 M sodium dihydrogen phosphate solution containing sodium lauryl sulfate 2% wt/wt as surfactant), 900 mL

Analysis method: High performance liquid chromatography

Detection wavelength: 247 nm

Mobile phase: methanol:0.025 M sodium dihydrogen phosphate buffer (pH=4.0)=67:33 (pH=4.0)

Column: stainless column (having an inner diameter of 4.6 mm and a length of 250 mm) packed with octadecyl silyl silica gel.

Flow rate: 1.5 mL/min.

As seen in FIG. 6, when the dissolution profile test was performed under the following conditions, the atorvastatin ingredient in the preparation of Example 30 showed a dissolution profile substantially equal to that of the control drug Lipitor. On the other hand, the diltiazem ingredient, which is a pharmacologically active ingredient of the delayed-release compartment in the preparation of Example 30, showed about 2 hour-delayed dissolution initiation as compared to that of the control drug Cardizem LA. That is, in the case of the diltiazem hydrochloride/atorvastatin multi-layered tablets of Example 30, dissolution rates of the diltiazem ingredient were all within 0.5% until 3 hours after the test began, which were lower than those of the control drugs (about 6%).

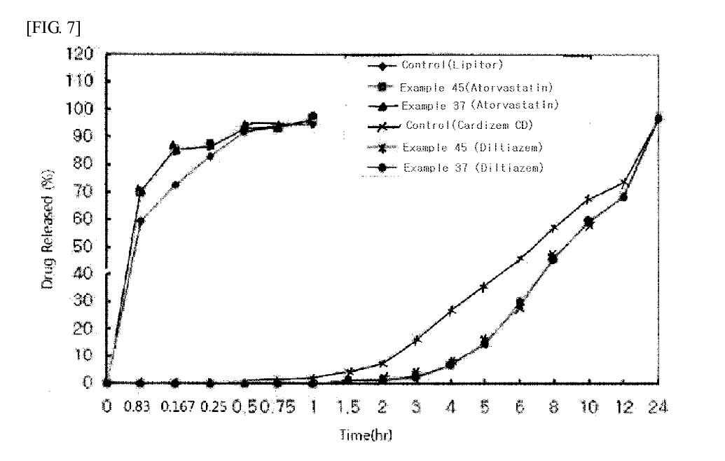

As can be seen in FIG. 7, the atorvastatin ingredient in the preparations of Examples 37 and 45 showed a dissolution profile substantially equal to that of the control drug Lipitor. On the other hand, the diltiazem ingredient, which is a pharmacologically active ingredient of the delayed-release compartment in the preparations of Examples 37 and 45, showed delayed dissolution initiation as compared to that of the control drug Cardizem CD. In the case of the diltiazem hydrochloride/atorvastatin preparations of Examples 37 and 45, dissolution rates of the diltiazem ingredient were all within 3% until 3 hours after the test began, which were far lower than those of the control drugs (about 17%).

As described above, the diltiazem hydrochloride/atorvastatin tablets and capsules according to the present invention exhibit delayed initiation of diltiazem release after the completion of atorvastatin release unlike the control drugs (i.e., diltiazem single drugs), and therefore diltiazem may be less likely to be metabolized in the liver ahead of atorvastatin.

### INDUSTRIAL APPLICABILITY

As described above, the present invention provides a pharmaceutical preparation which is designed based on chronotherapeutics and xenobiotics for maximizing therapeutic effects and for pharmacodynamically improving side effects that may occur upon combination use of different drugs. The combination product of the present invention comprises, as active ingredients, a non-dihydropyridine calcium channel blocker and a statin-based lipid-reducing agent, which are affected by the same cytochrome P450 enzyme or enzyme activity. At the same time, the combination comprises a release-controlling material, which may control the time when the active ingredients are released in a body, such that the pharmacologically active ingredients may be released at different times and different release rates in the body.

Therefore, the preparation of the present invention provides synergistic effects through combined administration of a non-dihydropyridine calcium channel blocker and an HMG-CoA reductase inhibitor, and induces the time-dependent absorption, metabolism and action mechanism of individual drugs through the controlled release thereof to avoid competitive antagonism between drugs, thus maximizing the effects of each pharmacologically active ingredient while minimizing side effects, for example, the risk of myopathy, and substantially increasing the compliance of patients by taking one tablet once a day.

1. A pharmaceutical preparation comprising a prior-release compartment containing a hydroxymethylglutaryl-CoA (HMG-CoA) reductase inhibitor as a pharmacologically active ingredient, and a delayed-release compartment containing a non-dihydropyridine calcium channel blocker as a pharmacologically active ingredient.

2. The pharmaceutical preparation according to claim 1, wherein the HMG-CoA reductase inhibitor includes at least one selected from the group consisting of simvastatin, lovastatin, atorvastatin, pitavastatin, rosuvastatin, fluvastatin, pravastatin, isomers thereof and pharmaceutically acceptable salts thereof.

3-4. (canceled)

5. The pharmaceutical preparation according to claim 1, wherein the non-dihydropyridine calcium channel blocker includes at least one selected from the group consisting of diltiazem, verapamil, gallopamil, cinnarizine, flunarizine, isomers thereof and pharmaceutically acceptable salts thereof.

6. (canceled)

7. The pharmaceutical preparation according to claim 1, wherein the HMG-CoA reductase inhibitor includes one selected from the group consisting of simvastatin, lovastatin, atorvastatin, pravastatin, isomers thereof and pharmaceutically acceptable salts thereof, and the non-dihydropyridine calcium channel blocker includes one selected from the group consisting of diltiazem, verapamil, isomers thereof and pharmaceutically acceptable salts thereof.

8-9. (canceled)

10. The pharmaceutical preparation according to claim 1, wherein the HMG-CoA reductase inhibitor includes one selected from the group consisting of simvastatin, isomers thereof and pharmaceutically acceptable salts thereof, and the non-dihydropyridine calcium channel blocker includes diltiazem, an isomer thereof or a pharmaceutically acceptable salt thereof.

11. (canceled)

12. The pharmaceutical preparation according to claim 1, wherein the HMG-CoA reductase inhibitor is released within one hour more than 85% of a total amount of the HMG-CoA reductase inhibitor in the preparation after an initiation of the release thereof.

13. The pharmaceutical preparation according to claim 1, wherein the release of the non-dihydropyridine calcium channel blocker is initiated 2 hours after the release of the HMG-CoA reductase inhibitor is initiated and is finished within 24 hours.

14. The pharmaceutical preparation according to claim 1, wherein the non-dihydropyridine calcium channel blocker is released within 6 hours less than 40% of a total amount of the non-dihydropyridine calcium channel blocker in a unit preparation after the release of the HMG-CoA reductase inhibitor is initiated.

15-16. (canceled)

17. The pharmaceutical preparation according to claim 1, wherein the delayed-release compartment includes at least one release-controlling material selected from an enteric polymer, a water-insoluble polymer, a hydrophobic compound, a hydrophilic polymer and a mixture thereof.

18-19. (canceled)

20. The pharmaceutical preparation according to claim 17, wherein the enteric polymer includes one selected from the group consisting of an enteric cellulose derivative, an enteric acrylic acid copolymer, an enteric maleic acid copolymer, an enteric polyvinyl derivative, and a mixture thereof.

21. The pharmaceutical preparation according to claim 20, wherein the enteric cellulose derivative includes at least one selected from the group consisting of hydroxypropylmethylcellulose acetate succinate, hydroxypropylmethylcellulose phthalate, hydroxymethylethylcellulose phthalate, cellulose acetate phthalate, cellulose acetate succinate, cellulose acetate maleate, cellulose benzoate phthalate, cellulose propionate phthalate, methylcellulose phthalate, carboxymethylethylcellulose, ethylhydroxyethylcellulose phthalate, methylhydroxyethylcellulose and a mixture thereof; the enteric acrylic acid copolymer includes at least one selected from the group consisting of a styrene/acrylic acid copolymer, a methyl acrylate/acrylic acid copolymer, an acrylic acid/methyl methacrylic acid copolymer, a butyl acrylate/styrene/acrylic acid copolymer, a methacrylic acid/methyl methacrylate copolymer, a methacrylic acid/ethyl acrylate copolymer, a methyl acrylate/methacrylic acid/octyl acrylate copolymer and a mixture thereof; the enteric maleic acid copolymer includes at least one selected from the group consisting of a vinyl acetate/maleic anhydride copolymer, a styrene/maleic anhydride copolymer, a styrene/maleic monoester copolymer, a vinyl methyl ether/maleic anhydride copolymer, an ethylene/maleic anhydride copolymer, a vinyl butyl ether/maleic anhydride copolymer, an acrylonitrile/methyl acrylate/maleic anhydride copolymer, a butyl acrylate/styrene/maleic anhydride copolymer and a mixture thereof; and the enteric polyvinyl derivative includes at least one selected from the group consisting of polyvinylalcohol phthalate, polyvinylacetal phthalate, polyvinylbutyrate phthalate, polyvinylacetacetal phthalate and a mixture thereof.

22. (canceled)

23. The pharmaceutical preparation according to claim 17, wherein the water-insoluble polymer include at least one selected from the group consisting of polyvinyl acetate, a polymethacrylate copolymer, a poly(ethyl acrylate, methyl methacrylate) copolymer, a poly(ethyl acrylate, methyl methacrylate, trimethylaminoethyl methacrylate) copolymer, ethylcellulose, cellulose ester, cellulose ether, cellulose acylate, cellulose diacylate, cellulose triacylate, cellulose acetate, cellulose diacetate, cellulose triacetate and a mixture thereof.

24. (canceled)

25. The pharmaceutical preparation according to claim 23, wherein the water-insoluble polymer includes a poly(ethyl acrylate, methyl methacrylate, or trimethylaminoethyl methacrylate) copolymer.

26. (canceled)

27. The pharmaceutical preparation according to claim 17, wherein the hydrophobic compound includes at least one selected from the group consisting of fatty acids and fatty acid esters, fatty acid alcohols, waxes, inorganic materials, and mixtures thereof.

28. The pharmaceutical preparation according to claim 27, wherein the fatty acid or fatty acid ester includes at least one selected from the group consisting of glyceryl palmitostearate, glyceryl stearate, glyceryl behenate, cetyl palmitate, glyceryl monooleate, stearic acid and a mixture thereof; the fatty acid alcohol includes at least one selected from the group consisting of cetostearyl alcohol, cetyl alcohol, stearyl alcohol and a mixture thereof; the wax includes at least one selected from carnauba wax, beeswax, microcrystalline wax and a mixture thereof; and the inorganic material includes at least one selected from the group consisting of talc, precipitated calcium carbonate, calcium hydrogen phosphate, zinc oxide, titanium oxide, kaolin, bentonite, montmorillonite, veegum and a mixture thereof.

29. (canceled)

30. The pharmaceutical preparation according to claim 17, wherein the hydrophilic polymer includes at least one selected from the group consisting of saccharide, a cellulose derivative, gum, a protein, a polyvinyl derivative, a polymethacrylate copolymer, a polyethylene derivative, a carboxyvinyl copolymer and a mixture thereof.

31. The pharmaceutical preparation according to claim 30, wherein the saccharide includes at least one selected from the group consisting of dextrin, polydextrin, dextran, pectin and a pectin derivative, alginate, polygalacturonic acid, xylan, arabinoxylan, arabinogalactan, starch, hydroxypropyl starch, amylose, amylopectin and a mixture thereof; the cellulose derivative includes at least one selected from the group consisting of hydroxypropylmethylcellulose, hydroxypropylcellulose, hydroxymethylcellulose, hydroxyethylcellulose, methylcellulose, sodium carboxymethylcellulose, hydroxypropylmethylcellulose acetate succinate, hydroxyethylmethylcellulose and a mixture thereof; the gum includes at least one selected from the group consisting of guar gum, locust bean gum, tragacanth, carrageenan, acacia gum, arabic gum, gellan gum, xanthan gum and a mixture thereof; the protein includes at least one selected from gelatin, casein, zein and a mixture thereof; the polyvinyl derivative includes at least one the group consisting of selected from polyvinyl alcohol, polyvinyl pyrrolidone, polyvinylacetal diethylaminoacetate and a mixture thereof; the polymethacrylate copolymer includes at least one selected from the group consisting of a poly(butyl methacrylate, (2-dimethylaminoethyl) methacrylate, methyl methacrylate) copolymer, a poly(methacrylic acid, methyl methacrylate) copolymer, a poly(methacrylic acid, ethyl acrylate) copolymer and a mixture thereof; the polyethylene derivative includes at least one selected from polyethylene glycol, polyethylene oxide and a mixture thereof; and the carboxyvinyl polymer is carbomer.

32. (canceled)

33. The pharmaceutical preparation according to claim 1, wherein the pharmaceutical preparation is in a form of a two-phase matrix tablet including the delayed-release compartment and the prior-release compartment.

34. The pharmaceutical preparation according to claim 1, wherein the pharmaceutical preparation is in a form of a film-coated tablet including a tablet containing the delayed-release compartment and a film-coating layer containing an prior-release compartment enclosing the exterior of the tablet.

35. The pharmaceutical preparation according to claim 1, wherein the pharmaceutical preparation is in a form of a multi-layered tablet having a multi-layered structure including the delayed-release compartment and the prior-release compartment.

36. The pharmaceutical preparation according claim 1, wherein the preparation is in a form of a press coated tablet including an inner core containing the delayed-release compartment and an outer layer containing an prior-release compartment enclosing an outer surface of the inner core.

37. The pharmaceutical preparation according to claim 36, wherein the press coated tablet is an osmotic press coated tablet.

38. The pharmaceutical preparation according to claim 1, wherein the pharmaceutical preparation is in a form of a capsule including a particle, granule, pellet, or tablet containing the delayed-release compartment and a particle, granule, pellet, or tablet containing the prior-release compartment.

39. The pharmaceutical preparation according to claim 1, further comprising a coating layer on the outside of at least one of the delayed-release compartment and the prior-release compartment.

40. The pharmaceutical preparation according to claim 1, wherein the delayed-release compartment includes an osmo-regulator and the delayed-release compartment is coated by a semi-permeable membrane coating base.

41-42. (canceled)

43. The pharmaceutical preparation according to claim 1, wherein the preparation is in the form of a coated tablet further including a coating layer on the outside thereof.

44. The pharmaceutical preparation according to claim 1, wherein the preparation is in the form of a kit including the delayed-release compartment and the prior-release compartment.

45. The pharmaceutical preparation according to claim 1, wherein the preparation is administered in the evening.

46. A method for treating a cardiovascular disease, comprising administering a pharmaceutical preparation including a prior-release compartment containing a hydroxymethylglutaryl-CoA (HMG-CoA) reductase inhibitor and a delayed-release compartment containing a non-dihydropyridine calcium channel blocker to a mammal.

47. The pharmaceutical preparation according to claim 1, wherein the delayed-release compartment includes at least one selected from the group consisting of polyethylene oxide, polyvinyl acetate, a poly(ethyl acrylate, methyl methacrylate, trimethylaminoethyl methacrylate) copolymer, a polymethacrylate copolymer, a polymethyl methacrylate ethyl acrylate copolymer, a carboxyvinyl polymer, hydroxypropylmethylcellulose phthalate, and titanium oxide.
